(19) United States
(12) Patent Application Publication
(10) Pub. No.: US 2009/0163415 A1
(43) Pub. Date: Jun. 25, 2009

(54) NT-proBNP, proBNP AND BNP IMMUNOASSAYS, ANTIBODIES AND STABLE STANDARD

(75) Inventors: Alexei G. KATRUKHA, Turku (FI); Karina R. SEFERYAN, Moscow (RU); Alexander G. SEMENOV, Moscow (RU); Natalia N. TAMM, Moscow (RU); Vladimir L. FILATOV, Moscow (RU); Alexander B. POSTNIKOV, Moscow (RU)

Correspondence Address: BIRCH STEWART KOLASCH & BIRCH, PO BOX 747, FALLS CHURCH, VA, 22040-0747, US

(73) Assignee: Hytest Ltd., Turku (FI)

(21) Appl. No.: 12/275,324

(22) Filed: Nov. 21, 2008

Related U.S. Application Data
(63) Continuation-in-part of application No. PCT/FI2007/050298, filed on May 25, 2007.
Application No. PCT/FI2007/050298 is a continuation-in-part of application No. PCT/FI2007/050599, filed on Nov. 8, 2007.
(60) Provisional application No. 60/808,695, filed on May 26, 2006.
(60) Provisional application No. 60/865,397, filed on Nov. 10, 2006.

(30) Foreign Application Priority Data
Mar. 15, 2007 (FI) FI 20075178
Nov. 23, 2007 (FI) FI 20075834

(51) Int. Cl. A61K 38/16; C07K 16/18; G01N 33/68; C07K 14/00; G01N 33/566

(52) U.S. Cl. 514/12; 530/387.1; 436/15; 530/350; 436/501

(57) ABSTRACT

The present invention provides antibodies against glycosylated proBNP and NT-proBNP. The antibodies are suitable for precise immunodetection of both of the proteins in human blood. The glycosylated forms of proBNP and NT-proBNP may be utilized as an antigen for antibody generation as well as a calibrator or immunological standard in different types of immunoassays. The invention thus also relates to a stable standard or calibrator pro-Brain Natriuretic Peptide (proBNP) preparation for use in a method for detecting BNP immunoreactivity in a sample, the preparation comprising glycosylated proBNP or a fragment thereof. In addition, the present invention is directed to an assay for precisely detecting the NT-proBNP circulating in a patient's blood, wherein the level of glycosylation of the proBNP molecule is exploited. Therapeutic applications are also contemplated.

This application is a continuation-in-part of PCT International Application PCT/FI2007/050599 which was filed on Nov. 8, 2007, and which claims priority under 35 U.S.C. § 119(a) on Finnish Application No. 20075178 filed on Mar. 15, 2007, and under 35 U.S.C. § 119(e) on U.S. Provisional Application No. 60/865,397 filed on Nov. 10, 2006. This application is also a continuation-in-part of PCT International Application PCT/FI2007/050298 which was filed on May 25, 2007, and which claims priority under 35 U.S.C. § 119(e) on U.S. Provisional Application No. 60/808,695 filed on May 26, 2006. This application also claims priority on Finnish Application No. FI 20075834 which was filed on Nov. 23, 2007. The entire contents of all of the above-identified applications are hereby incorporated by reference.

### FIELD OF THE INVENTION

The present invention relates to detection of pro-Brain Natriuretic Peptide (proBNP) and proBNP-derived peptides BNP and NT-proBNP. The stable glycosylated form of proBNP may be used as a standard or calibrator in immunoassays measuring BNP immunoreactivity, and as an antigen for antibody generation. The present invention thus also provides antibodies to the glycosylated forms of proBNP and NT-proBNP. Particular epitopes in these proteins are provided as well. Antibodies specific to these particular epitopes are suitable for precise immunodetection, i.e. determination of the presence and/or quantification of the amount, of both of the proteins in human blood. Furthermore, the present invention relates to assays for detecting or quantifying NT-proBNP in a patient sample, wherein the level of glycosylation of the proBNP molecule is exploited. Therapeutic applications of proBNP nucleic acid or amino acid sequences or Thr71 mutations thereof are also contemplated.

### BACKGROUND OF THE INVENTION

The pro-form of brain natriuretic peptide (proBNP) as well as BNP and N-terminal fragment of proBNP (NT-proBNP) are recognized markers of congestive heart failure (CHF) and left ventricle dysfunction. They are also used for risk stratification in patients with various cardiac pathologies, and therapy monitoring in patients with CHF. BNP is a peptide hormone with natriuretic, vasodilatory and renin inhibitory properties (reviewed in Mair et al. 2001; Cowie et al., 2002 and Pandey, 2005). BNP belongs to a family of structurally similar peptide hormones. BNP molecule is composed of 32 amino acid residues with a disulfide bond located between the residues Cys₁₀ and Cys₂₆. BNP is released from the heart and can be detected in blood by immunological methods. A synthetic form of BNP, i.e., BNP-32, is commonly used as a standard in immunoassays for measuring the blood concentration of BNP. Multiple studies demonstrated significant instability of synthetic BNP-32 when spiked into plasma or buffer solutions. Peptide instability significantly compromises utilization of synthetic BNP-32 in immunoassays as a liquid calibrator or liquid standard.

NT-proBNP and BNP are the products of proteolytic processing of the precursor molecule preproBNP (FIG. 9). PreproBNP is composed of 134 aar and is synthesized in cardiac myocytes. Removal of signal peptide (aar 1-26) results in the appearance of proBNP molecule (aar 27-134). Subsequently, proBNP (108 aar) is cleaved by proteases forming two peptides—BNP (aar 77-108) and NT-proBNP (aar 1-76). Two proprotein convertases, furin and corin have been discussed in the literature as possible candidates responsible for proBNP processing (Sawada et al. 1997; Yan et al. 2000). While it is still uncertain which of these two enzymes is responsible for proBNP processing in cardiomyocytes, some other convertases cannot be excluded, either. The BNP molecule contains a cyclic structure formed by intrinsic disulfide bond formation between two Cys residues. Both BNP (the biologically active molecule) and NT-proBNP (the physiological activity, if any, is not clearly understood) are secreted into the bloodstream in equimolar amounts and circulate in human blood. The BNP concentration in the blood of healthy adults is about 25 pg/ml (Wu et al., 2004), whereas the NT-proBNP concentration is about 200 pg/ml (Luchner et al., 2002).

It has been established that proBNP synthesis increases in response to mechanical or neurohormonal stimulation of the heart and this increase led to increases of BNP and NT-proBNP concentrations in serum. Elevated levels of BNP and NT-proBNP in human blood are reported for patients with different cardiovascular pathologies, such as heart failure, left ventricular dysfunction, unstable angina and myocardial infarction. Blood concentrations of both analytes in heart failure patients correlate with the severity of disease. It has been reported that both concentrations are already elevated in asymptomatic patients during the very early stage of heart failure (NYHA I stage according to the New York Heart Association classification).

BNP and NT-proBNP measurements are useful for risk stratification of patients with different cardiac pathologies. It was demonstrated that patients with possible complications characteristically exhibited significantly higher BNP and NT-proBNP concentrations than patients without complications (Luchner et al., supra). For instance, in patients with acute coronary syndrome, measurements of both peptides helped to discriminate those who were at risk of developing new cardiac events. NT-proBNP measurements were helpful not only for disease diagnosis but also for monitoring the therapy administered to patients with heart failure (Troughton et al., 2000).

Different immunoassay methods for NT-proBNP measurement in human plasma have been described in literature. Immunoassays utilize monoclonal as well as polyclonal antibodies, specific to different parts of the human NT-proBNP molecule: epitope 1-13 (Hunt et al., 1997), 65-76 (Hughes et al., 1999), 1-12 and 65-76 (Karl et al., 1999), 8-29 (Biomedica assay), 1-21 and 39-50 (Roche Elecsys).

There is still no consensus regarding antibodies which should be used in the NT-proBNP assays. However, some published data demonstrate that antibodies specific to the central regions of the NT-proBNP molecule are not able to recognize the analyte in human blood. Thus, Hughes et al. (1999) demonstrated that rabbit polyclonal antibodies specific to aar 37-49 did not recognize NT-proBNP in patients' blood in contrast to antibodies specific to aar 65-76. The reason of the observed absence of signal was not clear at that time.

Blood measurements of Brain natriuretic peptide (BNP) have been used as diagnostic and prognostic aids in congestive heart failure (CHF), and as a prognostic marker in acute coronary syndrome (ACS). In addition, BNP may prove useful as an aid in guiding medical therapy in patients with CHF. The growing interest in the clinical determination of BNP has led to the development of immunoassays that are suitable for fully automated, high-throughput clinical instruments with random access, e.g., the ADVIA Centaur BNP (Bayer Diagnostics) and the AxSYM BNP system (Abbott Laboratories).

The AxSYM BNP assay, like the ADVIA Centaur system and other approaches for assaying BNP, use conventional anti-BNP antibodies that specifically recognize an epitope in the mature 32-amino acid BNP. None of the known approaches shows sufficient assay accuracy and reproducibility as regards the standardization of the immunological measurement. Thus, a need exists in the art for a stable, reliable calibrator or standard for use in measuring BNP.

Recently, new proBNP immunoassay utilizing one antibody specific to the proBNP cleavage site and another to the BNP part was described (Giuliani et al., 2006).

Gel filtration (GF) studies in non-denaturating conditions demonstrated that NT-proBNP and proBNP immunoreactivities are represented in fractions of proteins with apparent molecular masses of 3- to 4-fold higher than expected values. Seidler et al. (1999) assumed that the differences observed in molecular masses during chromatography in different conditions are due to oligomerization of NT-proBNP and proBNP molecules in human blood. However, this hypothesis was rejected by Crimmins (2005) who showed that synthetic NT-proBNP does not form oligomers in vitro. Consequently, at that time the form in which NT-proBNP circulates in human blood had not been defined, and there was no explanation to the abnormalities as observed.

Consequently, information about biochemical properties of proBNP and NT-proBNP in human blood could significantly affect the current approach to proBNP and NT-proBNP measurement. It was recently shown in the art that endogenous proBNP is glycosylated. According to Schellenberger et al. (2006) proBNP expressed in mammalian CHO cell line contains several sites of O-glycosylation: Thr36, Ser37, Ser44, Thr48, Ser53, Thr58, Thr71. For recombinant protein Thr36 and Thr58 were defined as sites of partial glycosylation, and the rest of the residues mentioned as sites of complete glycosylation. On the other hand, the present inventors have demonstrated that NT-proBNP in human blood is also glycosylated and that glycosylation negatively influences the recognition of the NT-proBNP by antibodies specific to the central part of the molecule (Seferian et al., 2008).

### SUMMARY OF THE INVENTION

ProBNP and NT-proBNP, circulating in human blood, are described in literature as polypeptides consisting of 108 and 76 amino acid residues, respectively (molecular masses about 11.9 and 8.46 kDa). The concentration of proBNP in patient's blood is significantly lower than the concentration of NT-proBNP. According to our recent studies the concentration of proBNP in blood is about 10-20% of that of NT-proBNP. Seidler et al. (1999) demonstrated that in gel filtration studies (non-denaturing conditions) NT-proBNP and proBNP have anomalous mobility and their apparent molecular weights are about 30-40 kDa. The authors suggested that in human blood both proteins are present in homo-oligomeric forms. In our studies we have demonstrated that in blood both proteins have apparent molecular weight (gel filtration studies, Superdex 75 10/300 GL column) of about 30 kDa.

First of all, we generated a panel of high affinity monoclonal antibodies specific to NT-proBNP (also cross-reacting with proBNP) epitopes, covering the whole sequence of the protein (FIG. 1). All MAbs were generated after immunization of mice with synthetic peptides corresponding to different fragments of the NT-proBNP molecule. All MAbs were tested in two-site combinations (sandwich immunofluorescent assays) with recombinant protein as well as with endogenous protein, isolated from a patient's blood. The studies revealed that antibodies specific to the central part of NT-proBNP (epitopes located in the region 28-60) did not properly recognize the antigen in human blood (FIG. 2). On the other hand, most of the antibodies with epitopes located on peptides 5-27 and 61-76 recognized antigens in human blood with the same efficiency as recombinant proteins.

By means of affinity chromatography (affinity column containing immobilized monoclonal antibodies, specific to different regions of NT-proBNP) we purified endogenous antigen from human blood and analyzed it in Western blotting. NT-proBNP and proBNP molecules were stained by several NT-proBNP-specific monoclonal antibodies (FIG. 5). In the tracks containing endogenous protein none of the tested monoclonal antibodies recognized any protein band with the same molecular mass as recombinant NT-proBNP. The major immunological activity was concentrated on a diffused zone in the area corresponding to the proteins with higher molecular masses (15-70 kDa).

Such wide diversity of NT-proBNP and proBNP forms (FIG. 5) seen in Western blotting studies can be explained by glycosylation of NT-proBNP and proBNP molecules circulating in human blood.

We have thus demonstrated that the major part of proBNP in human blood is glycosylated. It also seems that the major part of BNP immunoreactivity detected by immunological methods in human blood is found in the pro-form of BNP (proBNP). These findings establish the benefits of using proBNP as a standard (calibrator) in immunoassays. Stability studies revealed that recombinant glycosylated proBNP demonstrated significantly higher stability (at least 10- to 1000-fold and even more) being measured in BNP assays in comparison with synthetic BNP-32. The stability of recombinant glycosylated proBNP was significantly higher than the stability of a recombinant protein which was not glycosylated. Consequently, proBNP is superior to synthetic BNP-32 for use in immunoassays as a stable calibrator or standard.

Furthermore, as indicated above, the central part (amino acids from about 28 to about 60) of NT-proBNP circulating in human blood is glycosylated (FIG. 19A, yellow oval), whereas N- and C-terminal portions (FIG. 19A, blue ovals; corresponding to amino acids from about 5 to about 27, and from about 61 to about 76 of NT-proBNP, respectively) do not contain glycosylated sites. Consequently, antibodies specific to N- and C-terminal parts of NT-proBNP could be used for endogenous NT-proBNP immunodetection.

Consequently, while Schellenberger's (2006) data shows that among other sites (FIG. 19B), also Thr71 (FIG. 19B, pink oval) is glycosylated, the present inventors have now demonstrated that it is not glycosylation in general, but the glycosylation of one particular amino acid—Thr71—which is a crucial point for proBNP processing. Specific protease-dependent conversion of proBNP into NT-proBNP and BNP is thus dependent on whether Thr71 is glycosylated or not. This is why glycosylation of Thr71 is also crucial for proBNP stability.

Therefore:
if Thr71 is glycosylated, proBNP (or at least the major part of it) cannot be cleaved by protease(s) to form NT-proBNP and BNP. Glycosylation of Thr71 protects proBNP from protease-dependent cleavage. In this case MAbs specific to an epitope comprising Thr71 are not able to recognize endogenous proBNP (or at least the major part of it); and
if Thr71 is not glycosylated, proBNP (or at least the major part of it) is cleaved by proteases, forming NT-proBNP and BNP. MAbs specific to an epitope comprising Thr71 are able to recognize endogenous NT-proBNP.

This finding explains the fact that MAbs specific to the C-terminal part of NT-proBNP can recognize only endogenous NT-proBNP, but not endogenous proBNP.

Accordingly, the first aspect of the invention is drawn to an antibody that specifically recognizes an endogenous glycosylated NT-proBNP or proBNP, or a fragment thereof, and which does not recognize deglycosylated NT-proBNP or proBNP or a fragment thereof, or fragments of such antibodies. In addition, the invention provides an antibody that recognizes an endogenous glycosylated NT-proBNP or proBNP, or a fragment thereof, with higher affinity than the antibody or fragment recognizes a corresponding deglycosylated protein, or fragment thereof, of such antibodies. A related aspect of the invention provides an aptamer having the same specificity as an antibody described above. Further the above-described antibodies include an antibody that is a monoclonal antibody or fragment of a monoclonal antibody, as well as an antibody that is a polyclonal antibody or fragment of a polyclonal antibody. Also, the antibody may be a recombinant antibody or a fragment of a recombinant antibody.

Another aspect according to the invention provides for the use of an antibody or antibody fragment as described above in a diagnostic immunoassay method for qualitative or quantitative detection of NT-proBNP or proBNP or a fragment thereof. Related thereto is a diagnostic method for assaying NT-proBNP or proBNP or a fragment thereof in a patient's blood sample, comprising quantitative, semiquantitative or qualitative determination of the NT-proBNP or proBNP content of the sample using an antibody or an aptamer, each as described herein. The diagnostic method may further comprise preparing a calibration curve using as the standard a preparation of an endogenous glycosylated NT-proBNP or proBNP isolated from vertebrate tissue or body fluids, such as blood. Also, the diagnostic method may further comprise preparing a standard curve and comparing the value of the NT-proBNP or proBNP content determined to the standard curve. Exemplary diagnostic methods according to the invention are immunoassay methods. For example, a diagnostic immunoassay is a sandwich immunoassay method, using a first capture antibody and a second detection antibody.

Another aspect of the invention is directed to a diagnostic method for assaying NT-proBNP or proBNP in a sample of a patient comprising (a) deglycosylating endogenous NT-proBNP or proBNP contained in the sample; and (b) determining the NT-proBNP or proBNP content of the sample using an antibody or an aptamer specific to NT-proBNP or proBNP.

Yet another aspect is an NT-proBNP or proBNP standard or calibration preparation comprising a glycosylated NT-proBNP or proBNP or a fragment thereof. In some embodiments, the glycosylated NT-proBNP or proBNP or a fragment thereof is an endogenous NT-proBNP or proBNP; in some embodiments, the NT-proBNP or proBNP is isolated from vertebrate tissue or body fluids, such as blood. A related aspect of the invention provides an isolated glycosylated recombinant NT-proBNP or proBNP or a fragment thereof. In some embodiments, the glycosylated recombinant NT-proBNP or proBNP, or a fragment thereof, is glycosylated in vitro. In some embodiments, the glycosylated recombinant NT-proBNP or proBNP, or a fragment thereof, is produced in a cell culture or in a cell-free translation system.

Yet another aspect of the invention is a use of a molecule as described above, as an antigen for producing an antibody having the same specificity as an antibody described above.

In another aspect, the invention provides an immunoassay kit for diagnostic assay of NT-proBNP or proBNP or a fragment thereof in a patient sample, the kit comprising (a) a monoclonal or polyclonal antibody having the same specificity as an antibody described hereinabove; (b) a detectable label; and (c) a standard or calibrator preparation as described herein. An exemplary kit is an immunoassay kit for diagnostic assay of NT-proBNP or proBNP or a fragment thereof in a sample of a patient, the kit comprising (a) a first monoclonal capture antibody of the same specificity as an antibody described above; (b) a second monoclonal detection antibody of the same specificity as an antibody of claim 1 or 2, wherein the detection antibody is fluorescently labeled; and (c) a standard or calibration preparation according to any one of claims 7 to 12.

The fragments of proteins, antibodies or any other entities as referred to in this specification mean any structural and/or functional fragments of said entities, retaining the desired activity.

In addition, a further aspect of the invention is directed to a standard pro-Brain Natriuretic Peptide (proBNP) preparation for use in a method for detecting BNP (SEQ ID NO:4) or its fragments, or molecules comprising this sequence or part of it in a sample, wherein the standard preparation comprises an isolated or recombinant or synthetic proBNP having the amino acid sequence given in SEQ ID NO:1, or a fragment or modification thereof, in glycosylated form, in combination with at least one diluent. A fragment of proBNP is a functional polypeptide fragment of proBNP that retains the capacity to specifically bind to at least one antibody that specifically binds full-length proBNP. In one embodiment, a functional fragment will comprise amino acids 1-76 of SEQ ID NO:1. In some embodiments, a modification of proBNP, i.e. a modified proBNP is a polypeptide having a sequence identity of at least 90%, 95%, 98%, 99% or 99.9% identity to SEQ ID NO:1 or SEQ ID NO:2, or a polypeptide having a sequence that differs from SEQ ID NO:1 or SEQ ID NO:2 by the substitution of six or fewer amino acids (preferably, conservative amino acid substitutions), the insertion of six or fewer amino acids or the deletion of six or fewer amino acids. One of skill will be able to identify these and other polypeptides according to the invention using routine skills and procedures because each such polypeptide will exhibit a structure that is recognizably similar to SEQ ID NO:1 or SEQ ID NO:2 in being at least 90% identical in primary amino acid sequence, and in exhibiting the functional property of specifically binding to a binding partner, e.g., antibody, that specifically binds to a polypeptide having the sequence set forth in SEQ ID NO:1, SEQ ID NO:2, and/or SEQ ID NO:4.

In some embodiments the proBNP, a fragment thereof or a modified proBNP is a recombinant proBNP or an endogenous proBNP. In specific, the proBNP, a fragment thereof or a modified proBNP is contemplated as being glycosylated proBNP. Further, the proBNP or a fragment thereof or a modified proBNP may be expressed in eukaryotic cells, such as human cells. In some embodiments, the proBNP or a fragment thereof or a modified proBNP is a synthetic proBNP. The invention comprehends modified proBNP polypeptides that comprise at least one amino acid residue change in at least one out of 76 amino acid residues from the N-terminus of proBNP, i.e., in the N-terminal fragment of the amino acid sequence of proBNP. Exemplary amino acid modifications are described above.

Another aspect according to the invention is a use of a peptide selected from the group consisting of an isolated or recombinant or synthetic proBNP having the amino acid sequence given in SEQ ID NO:1, an isolated or recombinant or synthetic proBNP fragment and an isolated or recombinant or synthetic modified proBNP as a standard or calibrator, in a method for detecting BNP immunoreactivity in a sample. It is contemplated that in each of the uses according to the invention, any of the above-described peptides or preparations may be used, or an isolated full-length proBNP may be used. In certain embodiments of the use, the peptide is an endogenous proBNP isolated from human tissue or body fluids, such as human blood; in other embodiments, the proBNP is a recombinant proBNP peptide. The isolated or recombinant proBNP is preferably a glycosylated proBNP. Further, the peptide, e.g., glycosylated proBNP, may be expressed in eukaryotic cells (endogenously or recombinantly), such as human cells. As indicated, the peptides employed in the uses according to the invention may be synthetic peptides, such as a synthetic proBNP. A synthetic peptide is a peptide constructed by the hand of man outside of a cell using any conventional technique for synthesizing peptides.

Another aspect of the invention is a diagnostic immunoassay method for determining BNP having the amino acid sequence as given in SEQ ID NO:4 or its fragments or molecules comprising this sequence or part of it in a serum, plasma, whole blood or other body fluid sample of a patient, comprising (a) measuring BNP immunoreactivity in a sample; (b) pre-paring a standard curve using as a standard a polypeptide selected from the group consisting of an isolated or recombinant or synthetic proBNP, an isolated or recombinant or synthetic proBNP fragment and an isolated or recombinant or synthetic modified proBNP; and (c) comparing the value of the BNP immunoreactivity obtained in step (a) to the standard curve produced in step (b) in order to quantify the BNP immunoreactivity in the sample.

Yet another aspect of the invention is drawn to an immunoassay kit for diagnostic measurement of BNP immunoreactivity in a sample of a patient, the kit comprising a monoclonal or polyclonal antibody specific to BNP; a detectable label; and a polypeptide selected from the group consisting of an isolated or recombinant or synthetic proBNP, an isolated or recombinant or synthetic proBNP fragment and an isolated or recombinant or synthetic modified proBNP.

Another aspect of the invention is a use of proBNP as described above as a standard or calibrator in a method for detecting BNP immunoreactivity in a sample. A related aspect of the invention is drawn to a method for generating a standard curve of immunoassay signal as a function of BNP amount, which is useful in diagnostic immunoassays.

In another aspect, the invention provides a diagnostic immunoassay method for assaying BNP in a serum sample of a patient. In such a method the BNP immunoreactivity in the sample is determined, and the value of the BNP so obtained is compared to a standard curve which has been prepared using the standard or calibrator preparation of the invention. A diagnostic immunoassay is, for instance, a sandwich immunoassay method, using a first capture antibody and a second detection antibody.

A still further aspect of the invention is an immunoassay kit for diagnostic assaying of BNP in a serum sample of a patient. Such a kit comprises a monoclonal or polyclonal antibody specific to BNP, a detectable label, and the standard or calibrator preparation according to the invention.

In addition, we provide a new method for precise NT-proBNP measurements, as well as new tools for determination of the influence of glycosylation on proBNP processing.

The present invention thus also provides an immunoassay method utilizing one antibody specific to the epitope comprising Thr71 and another antibody specific to the N-terminal portion of NT-proBNP. The method of the invention can be used for the precise immunodetection of NT-proBNP separately from immunodetection of proBNP. All other MAb combinations would detect both molecules, thus misrepresenting the real concentration of NT-proBNP.

Furthermore, the invention provides an immunoassay method measuring proBNP concentration by utilizing one antibody specific to the N-terminal portion of proBNP (FIG. 19B, red oval; corresponding to residues about 1 to about 31 of SEQ ID NO:1) and another antibody specific to the BNP portion of proBNP (FIG. 19B, green oval; corresponding to SEQ ID NO:4) together with NT-proBNP measurement by the method described above. The concentrations of the two analytes measured by two assays as described are subsequently compared.

A method of treatment of a disorder, comprising administering to a patient in need of such treatment an efficacious amount of mutated proBNP, from which Thr71 has been deleted or in which Thr71 residue has been changed to any other amino acid residue, is also contemplated. A further object of the invention is a method of treatment of a disorder, comprising administering to a patient in need of such treatment a nucleic acid encoding proBNP amino acid sequence, from which Thr71 has been deleted or in which Thr71 residue has been changed to any other amino acid residue.

A still further object of the invention is a recombinant proBNP polypeptide, wherein the Thr71 residue is glycosylated, preferably expressed in eukaryotic cells. Furthermore, a recombinant or synthetic proBNP polypeptide having at least one modification in any of its amino acid residues 66 to 76 is within the scope of the invention. The most preferable modification is the proBNP polypeptide having the modification Thr71Ala (SEQ ID NO:5).

Other features and advantages of the present invention will be better understood by reference to the brief description of the drawings and the detailed description that follow.

### BRIEF DESCRIPTION OF DRAWINGS

The accompanying drawings are provided to help further in describing the invention, which drawings are the following:

FIG. 1 illustrates epitope map of NT-proBNP-specific monoclonal antibodies used in the primary studies.

FIG. 2 illustrates specificities of different MAbs to endogenous NT-proBNP (antigen from HF patients' plasma). The results are presented as a ratio of signals in plasma to signals with recombinant NT-proBNP standard preparation in different two-site MAbs combinations. One MAb in such combination was able to interact effectively with endogenous NT-proBNP (MAb 24E11, epitope 67-76 or MAb 13G12, epitope 13-20), whereas another one was out of the set of antibodies, specific to different regions of NT-proBNP molecule. The concentration of recombinant NT-proBNP was the same as that of the endogenous antigen, determined in HF patients' plasma by 15C4-13G12 assay.

FIG. 3 shows the calibration curve for the assay 15C4-13G12 and dilution curves for two individual plasma samples. Recombinant NT-proBNP (expressed in E. coli, HyTest) reconstituted in pooled normal human plasma was used as a calibrator.

FIG. 4 illustrates gel filtration studies (Superdex 75 10/300 GL column) of four plasma samples from HF patients. NT-proBNP immunoreactivity in the fractions was quantified by sandwich immunoassay utilizing monoclonal antibodies 15C4 (epitope 63-71) and 13G12 (epitope 13-20) recognizing endogenous NT-proBNP.

FIG. 5 illustrates Western blotting studies of affinity-purified endogenous NT-proBNP. Tracks 1, 4: recombinant NT-proBNP (50 ng/track), tracks 2, 5: recombinant proBNP (50 ng/track), tracks 3, 6: endogenous NT-proBNP purified from human plasma (200 ng/track). For immunostaining MAbs 15F11 (epitope 13-24)—tracks 1-3 or MAb 11D1 (epitope 31-39)—tracks 446 were used.

FIG. 6 illustrates the specificities of different monoclonal antibodies to endogenous NT-proBNP (black columns) or to endogenous NT-proBNP after deglycosylation—treatment with O-glycosidase and sialidase—(grey columns). The results are represented as ratio of signals (endogenous/recombinant) in different two-site MAb combinations. Three forms of NT-proBNP a) recombinant (non-glycosylated), b) endogenous, extracted from HF human plasma and c) endogenous, extracted from plasma and treated with enzymes in same concentrations were tested by sandwich immunoassays utilizing different monoclonal antibodies. One MAb in such immunoassay was specific to the epitope that is not affected by glycosylation (MAbs 24E11, epitope 67-76 or MAb 13G12, epitope 13-20), whereas the other one was one out of the set of antibodies specific to different regions of NT-proBNP molecule.

FIG. 7 illustrates gel filtration studies (Superdex 75 10/300 GL column) of endogenous NT-proBNP extracted from plasma (uniform line) and endogenous NT-proBNP treated with O-glycosidase and sialidase (dotted line). NT-proBNP immunoreactivitics in the fractions were quantified by two sandwich immunoassays 15C4-13G12 (▴) and 11D1-13G12 (▪). Immunoassay 15C4-13G12 is not sensitive to glycosylation. Immunoassay 11D1-13G12 is sensitive to glycosylation.

FIG. 8 illustrates the Western blotting studies of affinity-purified NT-proBNP before and after deglycosylation. Tracks 1, 5: recombinant NT-proBNP (E. coli, 50 ng per track); tracks 2, 6: recombinant proBNP (E. coli, 50 ng per track); tracks 3, 7: affinity-purified endogenous NT-proBNP (200 ng per track); tracks 4, 8: affinity-purified endogenous NT-proBNP after deglycosylation (200 ng per track). MAb 5F11 (epitope 13-24)—tracks 1-4 or MAb 11D1 (epitope 31-39)—tracks 5-8 were used for the antigen immunostaining.

FIG. 9 provides a schematic of preproBNP processing in vivo.

FIG. 10 provides titration curves of different monoclonal antibodies (MAbs) using ELISA. Antigen: Human recombinant NT-proBNP, quantity: 0.01 μg/well.

FIG. 11 schematically illustrates epitope locations of monoclonal antibody binding sites suitable for use in, e.g., sandwich immunoassays of NT-proBNP immunoreactivity.

FIG. 12 shows calibration curves of suitable monoclonal antibodies suitable for use in the processes according to the invention. The assays were one-step fluoroimmunoassays performed in streptavidin-coated plates. Monoclonal antibodies used for capture: biotinylated 15F11 or 15C4 from Hytest Ltd. Monoclonal antibodies used for detection: Eu-labeled 24E11, 29D12, 13G12 or 18H5, all from Hytest Ltd. Sample volume: 50 μg, Antigen: human recombinant NT-proBNP, Incubation time: 30 minutes at room temperature.

FIGS. 13A and 13B disclose stability studies of endogenous NT-proBNP. Pooled blood samples from patients with heart failure were incubated at 4° C. (-▪-) and at room temperature (-♦-) for 72 hours.

FIG. 14 provides a calibration curve for a proBNP sandwich immunoassay. Capture antibody was MAb 50E1 (BNP specific; Hytest Ltd.), detection antibody was MAb 16F3 (NT-proBNP specific; Hytest Ltd.). A one-step assay was performed in streptavidin-coated plates. Biotinylated monoclonal antibodies for capture and Eu-labeled monoclonal antibodies for detection were used. Sample volume: 50 μg, Antigen: human recombinant proBNP, Incubation time: 30 minutes at room temperature.

FIG. 15 discloses the detection of human recombinant NT-proBNP in Western blots using different monoclonal antibodies after Tricine-SDS gel electrophoresis. Lane 1: MAb 5B6, Lane 2: 15F11, Lane 3: 11D1, Lane 4: 15D7, Lane 5: 13C1, Lane 6: 24E11. All antibodies from Hytest Ltd. NT-proBNP quantity: 2.5 μg/well.

FIG. 16 is a gel electrophoretogram of Tricine-SDS-PAGE fractionating a sample of recombinant proBNP under reducing conditions. Lane 1: Molecular weight standards (BioRad); Lane 2: rec proBNP (recombinant), 3 μg; Lane 3: rec NT-proBNP (recombinant), 3 μg. The gel was stained with Coomassie brilliant blue R-250.

FIG. 17 shows stabilities of different antigens in BNP immunoreactivity measurement. Incubation at 4° C.

FIG. 18 shows stabilities of different antigens in BNP immunoreactivity measurement. Incubation at 25° C.

FIG. 19A shows the glycosylation pattern of NT-proBNP circulating in human blood. The central part (amino acids from about 31 to about 60) of NT-proBNP is glycosylated (yellow oval), whereas N- and C-terminal portions do not contain glycosylated sites (blue ovals; corresponding to amino acid residues of about 6 to about 29 and about 61 to about 76, respectively).

FIG. 19B shows the glycosylation sites of proBNP disclosed by Schellenberger et al. (2006).

FIG. 20. Two proBNP assays recognize different amounts of proBNP in human blood. Assay 1 utilizes MAb 13G12 (epitope 13-20) as a capture, whereas Assay 2 utilizes as a capture MAb 21E6 (epitope 67-73).

FIG. 21. NT-proBNP/proBNP ratios in culture media of cells transfected by a plasmid containing wild type and Thr71Ala genes.

FIG. 22. Furin-dependent degradation of proBNP expressed in HEK 293 cells and in E. coli.

FIG. 23. Immunoreactivity profile of proBNP expressed in HEK 293 or CHO-K1 cells transiently transfected with proBNP expressing plasmid.

FIGS. 24A and 24B.

24(A): Immunochemical activity profiles of endogenous proBNP and NT-proBNP. Concentration of endogenous proBNP measured by reference assay 1D4₁₃₋₂₄-24C5₈₇₋₉₈ (§) was accepted as 100%. Concentration of endogenous NT-proBNP measured by reference assay 15C4₆₃₋₇₁-13G12₁₃₋₂₀ (§§) was accepted as 100%.

24(B): Comparison of endogenous proBNP immunoreactivity before and after enzymatic deglycosylation. Concentrations of endogenous proBNP in non-treated sample measured by reference assay 1D4₁₃₋₂₄-24C5₈₇₋₉₈ (§) was accepted as 100%. Results are expressed as mean±SD (n=3).

FIG. 25. Immunochemical activity profiles of recombinant (expressed in eukaryotic cell line HEK 293) proBNP and NT-proBNP. Concentrations of proBNP measured by reference assay 1D4₁₃₋₂₄-24C5₈₇₋₉₈ (§) was accepted as 100%. Concentrations of NT-proBNP measured by reference assay 15C4₆₃₋₇₁-13G12₁₃₋₂₀ (§§) was accepted as 100%. Results are expressed as mean±SD (n=3).

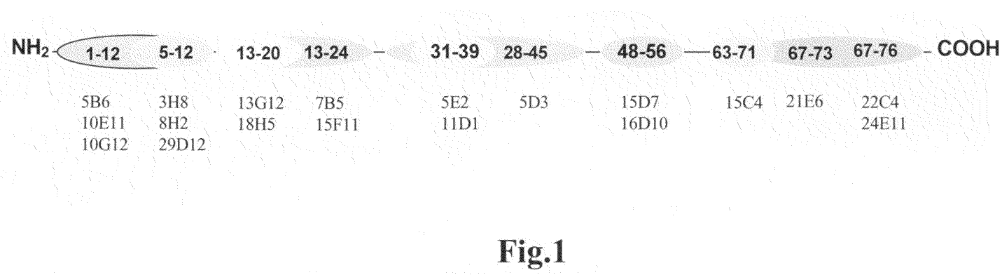

### DETAILED DESCRIPTION OF THE INVENTION

At first instance, we treated isolated NT-proBNP and proBNP with deglycosylation enzymes and analyzed protein preparations by means of sandwich immunoassay, gel filtration HPLC and Western blotting. These experiments, described in detail in the Examples 1 to 13 below, yielded the following results:

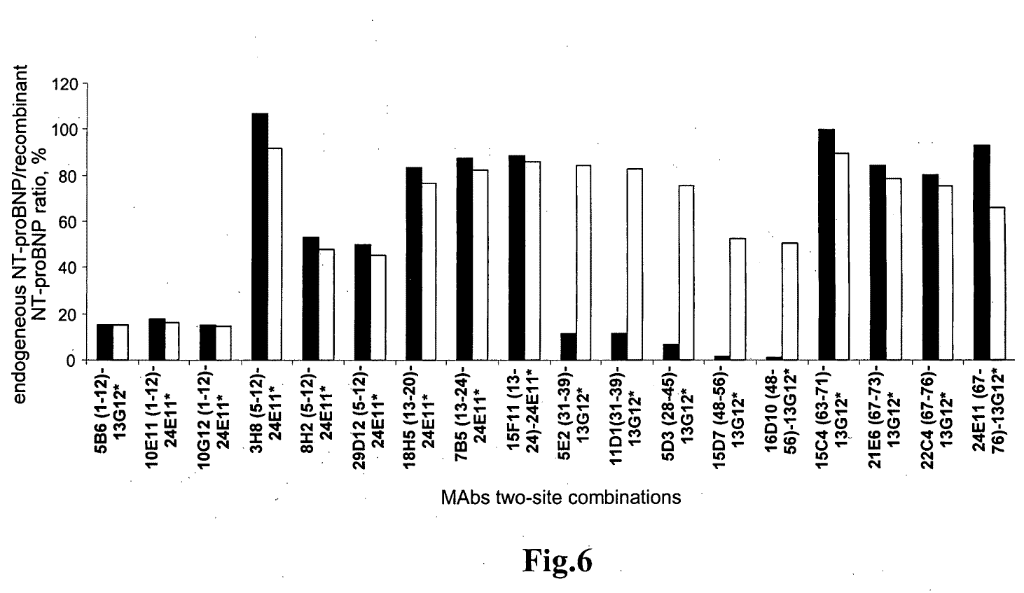

Sandwich immunoassay: After deglycosylation, monoclonal antibodies specific to the central region of NT-proBNP molecule (antibodies that do not recognize native endogenous antigen) were able to recognize endogenous protein with the same efficiency as the recombinant (non-glycosylated) form (FIG. 6).

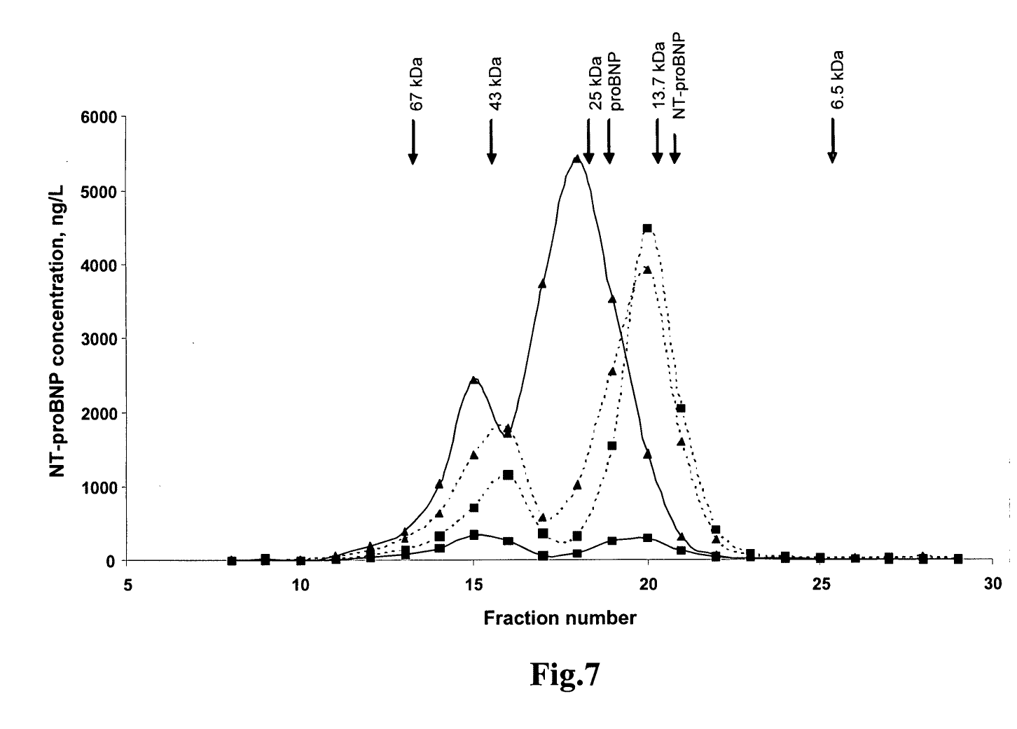

HPLC gel filtration chromatography: After deglycosylation, a shift of the peak of immunological activity towards the peak of recombinant protein (i.e. towards the proteins with lower molecular mass) was observed (FIG. 7).

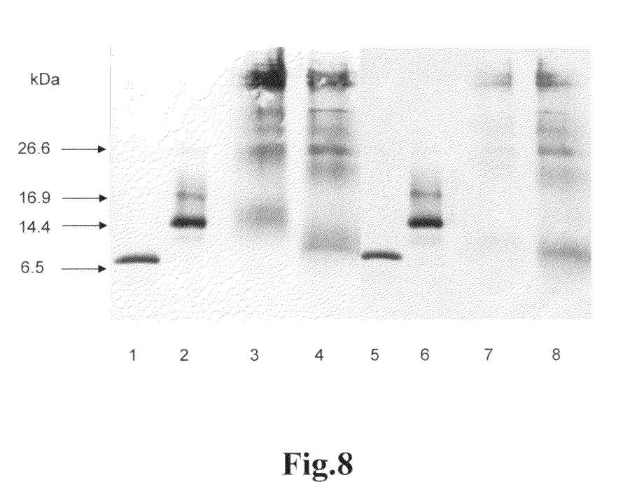

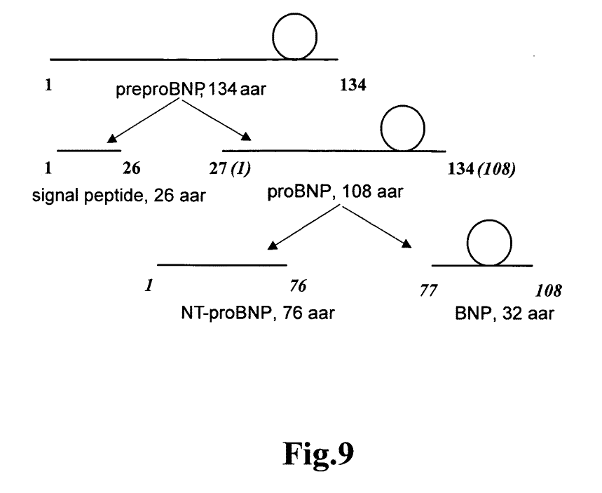

Western blotting:
1) After deglycosylation, the appearance of the protein band revealing NT-proBNP immunological activity on the same level as recombinant NT-proBNP was observed.
2) Antibodies specific to the central part of the NT-proBNP molecule (antibodies that do not recognize native endogenous antigen) were able to recognize this band (endogenous protein after deglycosylation) (FIG. 8).

### Conclusions

It is clearly disclosed in the present specification that in human blood NT-proBNP and proBNP do not exist as simple polypeptide chains, as was considered before, but as glycoproteins. This means that mono- or polyclonal antibodies for assays designed to detect one or both of the molecules in human blood should either recognize those parts of the molecules that are not affected by glycosylation, or they should recognize the glycosylated part of the molecule.

Glycosylated forms of both of the proteins should be used for the preparation of standards and calibrators for such assays.

Glycosylated forms of the antigens should be used for animal immunization to obtain antibodies specific to the glycosylated part of the molecule.

BNP and NT-proBNP measurements have received interest as a basis for a method for evaluating patients with suspected heart failure. Early diagnosis of heart failure is important for early drug intervention and might diminish the morbidity and mortality associated with advanced stages of these diseases.

At present both BNP and NT-proBNP are used in clinical practice and there is no agreement between clinicians as to which one is preferable. There is a correlation between NT-proBNP and BNP concentrations and apparently the diagnostic and prognostic values are similar. The NT-proBNP concentration in the blood of patients, however, is 2-5 times higher than the BNP concentration. This fact is explained by the longer half-life of NT-proBNP in patients' blood in comparison with BNP. Thus, the NT-proBNP immunoassay reveals higher sensitivity because of the rather high signal-to-noise ratio characteristic of NT-proBNP assays.

High-affinity monoclonal antibodies (see Table 1, listing suitable antibodies from Hytest Ltd.) that are specific to different epitopes of the NT-proBNP molecule are known. These antibodies, as well as anti-BNP antibodies, are useful in the development of quantitative sandwich NT-proBNP (or BNP, or total BNP+proBNP) immunoassays, immunodetection of antigen in direct ELISA, and Western blotting. Characterizing information relating to the antibodies listed in Table 1 follows. For the anti-NT-proBNP monoclonal antibodies (Hytest Ltd.):

| | |
|---|---|
| Host animal: | Balb/c mice |
| Cell line used | Sp 2/0 |
| for fusion: | |
| Antigen: | Synthetic peptides corresponding to amino acid |
| | residues 1-12, 13-27, 28-45, 46-60, 61-76 of the |
| | NT-proBNP sequence, |
| | conjugated to carrier protein. |
| Specificity: | Human NT-proBNP and proBNP |
| Purification method: | Protein A affinity chromatography |
| Presentation: | MAb solution in PBS with 0.1% sodium azide |

Hybridomas producing monoclonal antibodies were generated after immunization of Balb/c mice with synthetic peptides, corresponding to different parts of NT-proBNP (amino acid residues 1-12, 13-27, 28-45, 46-60, 61-76 of SEQ ID NO:3), conjugated to carrier protein. Precise epitope specificity of all MAbs was determined using a synthetic peptide library. All antibodies were checked for their abilities to recognize recombinant NT-proBNP (Hytest Ltd.) in direct ELISA, sandwich immunoassay, and Western blotting.

TABLE 1

| | Cat. | | | | |
|---|---|---|---|---|---|
| MAb | No. | Specificity | Sub-class | Epitope | Application |
| 5B6 | 4NT1 | Human | IgG1 | a.a.r. 1-12 | EIA, WB |
| | | NT-proBNP, | | | |
| | | proBNP | | | |
| 29D12 | 4NT1 | Human | IgG2a | a.a.r. 5-12 | EIA, Sandwich |
| | | NT-proBNP, | | | immunoassay, |
| | | proBNP | | | WB |
| 15F11 | 4NT1 | Human | IgG2b | a.a.r. 13-24 | EIA, Sandwich |
| | | NT-proBNP, | | | immunoassay, |
| | | proBNP | | | WB |
| 7B5 | 4NT1 | Human | IgG1 | a.a.r. 13-24 | EIA, Sandwich |
| | | NT-proBNP, | | | immunoassay, |
| | | proBNP | | | WB |
| 13G12 | 4NT1 | Human | IgG2a | a.a.r. 13-20 | EIA, Sandwich |
| | | NT-proBNP, | | | immunoassay, |
| | | proBNP | | | WB |
| 18H5 | 4NT1 | Human | IgG1 | a.a.r. 13-20 | EIA, Sandwich |
| | | NT-proBNP, | | | immunoassay, |
| | | proBNP | | | WB |
| 16F3 | 4NT1 | Human | IgG1 | a.a.r. 13-20 | EIA, Sandwich |
| | | NT-proBNP, | | | immunoassay, |
| | | proBNP | | | WB |
| 11D1 | 4NT1 | Human | IgG1 | a.a.r. 31-39 | EIA, WB |
| | | NT-proBNP, | | | |
| | | proBNP | | | |
| 16E6 | 4NT1 | Human | IgG1 | a.a.r. 34-39 | EIA, Sandwich |
| | | NT-proBNP, | | | immunoassay, |
| | | proBNP | | | WB |
| 15D7 | 4NT1 | Human | IgG1 | a.a.r. 48-56 | EIA, WB |
| | | NT-proBNP, | | | |
| | | proBNP | | | |
| 15C4 | 4NT1 | Human | IgG2b | a.a.r. 63-71 | EIA, Sandwich |
| | | NT-proBNP, | | | immunoassay, |
| | | proBNP | | | WB |
| 24E11 | 4NT1 | Human | IgG2a | a.a.r. 67-76 | EIA, Sandwich |
| | | NT-proBNP, | | | immunoassay, |
| | | proBNP | | | WB |
| 28F8 | 4NT1 | Human | IgG2a | a.a.r. 67-76 | EIA, Sandwich |
| | | NT-proBNP, | | | immunoassay, |
| | | proBNP | | | WB |

Some applications of these antibodies are described in items 1 to 4 below.

### 1. Direct ELISA

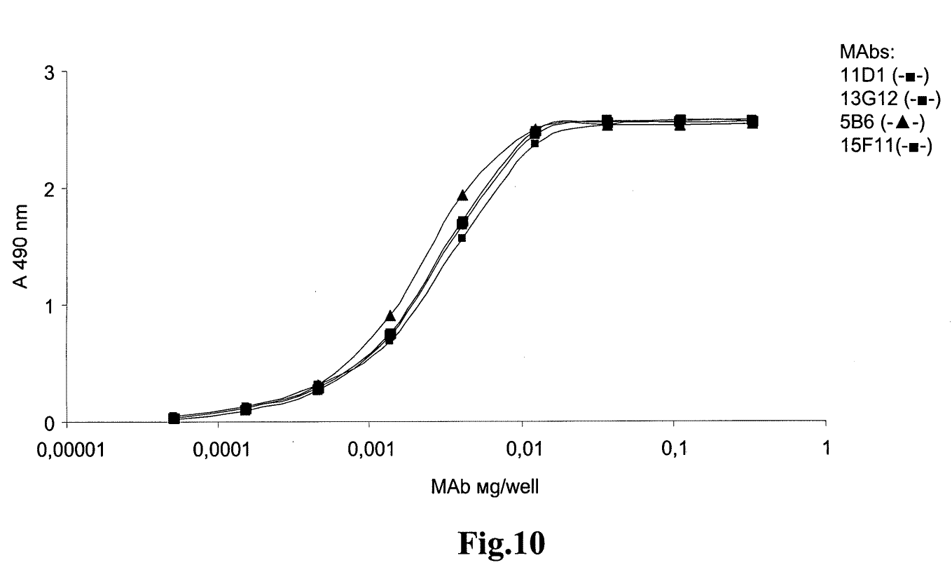

The monoclonal antibodies of Table 1 recognize recombinant human NT-proBNP and proBNP (Hytest Ltd.) in direct ELISA, and the results of ELISA experiments are shown in FIG. 10. The data shown in FIG. 10 pertains to four monoclonal antibodies, each of which demonstrates a titratable capacity to bind NT-proBNP.

### 2. NT-proBNP Quantitative Sandwich Immunoassays

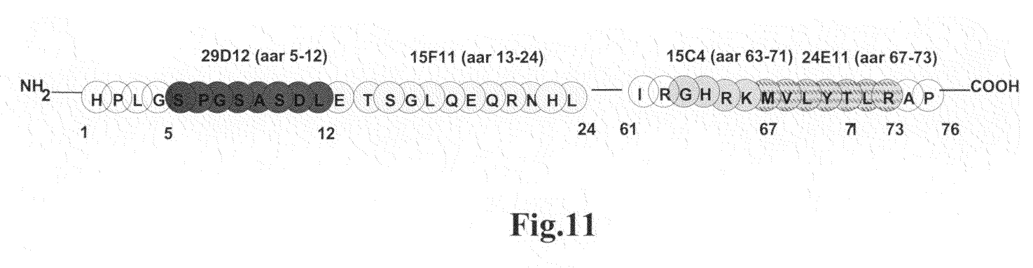

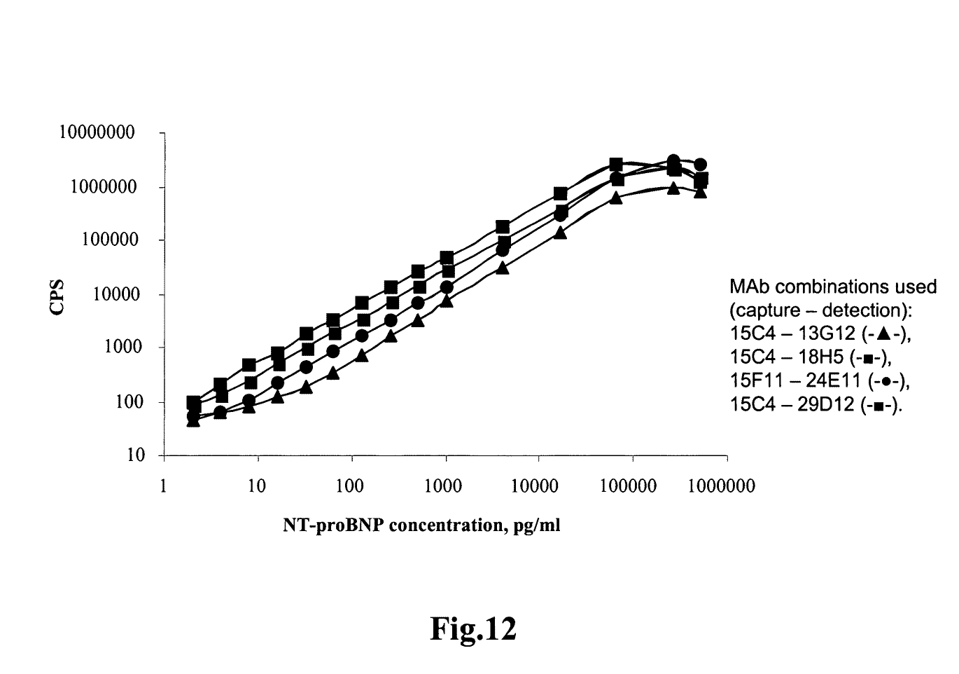

All MAbs were tested in sandwich fluoroimmunoassay as capture and detection antibodies with recombinant antigen and pooled blood samples from the patients with chronic heart failure. The experiments revealed that the majority of antibodies specific to the central part of the NT-proBNP molecule (a.a.r. 28-45 and a.a.r. 46-60) utilized in the immunoassays performed relatively poorly in detecting the antigen in human blood. Therefore, for precise quantitative NT-proBNP measurements from human blood, it is preferred that two-site antibody combinations utilizing antibodies specific to a.a.r. 5-12, 13-27 and 61-76 (FIG. 11 and FIG. 12) be used. Preferred antibody pairs (capture-detection) are:

### 15F11-24E11

### 15C4-29D12

### 15C4-13G12

### 15C4-18H5.

Each of these preferred antibody pairs demonstrates high sensitivity in antigen recognition (10-15 pg/ml) and good kinetics. All preferred sandwich MAb combinations were tested with blood samples from patients with heart failure, and each preferred MAb pair was able to detect antigen circulating in human blood.

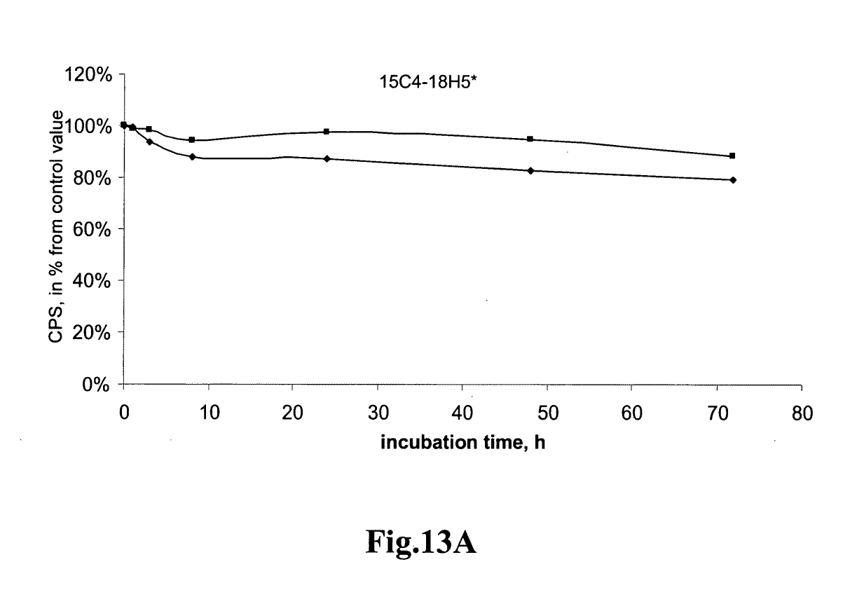

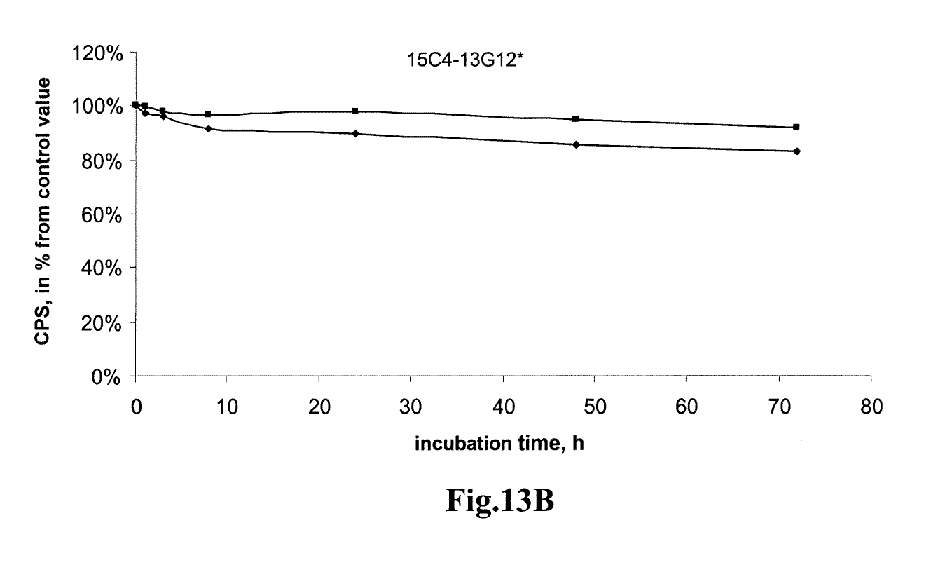

NT-proBNP is known as an unstable molecule with the least stability described for the very N- and C-terminal regions of peptide. The stability of endogenous NT-proBNP was assessed by incubating patients' blood samples at two temperatures (4° C. and 20° C.) for different time periods and measuring the immunological activity of the antigen in recommended sandwich immunoassays. The highest stability was obtained using two pairs of MAbs: 15C4-13G12 and 15C4-18H5 (FIG. 13). Insignificant changes in antigen activity (less than 10%) were observed after 72 hours of incubation at 4° C., and about 10-15% loss were seen after incubation of the sample after 24 hours at room temperature. Thus, serum samples can be stored by being refrigerated (or even maintained at room temperature) for a relatively long time prior to determination of NT-proBNP immunoreactivity levels.

### 3. ProBNP Quantitative Sandwich Immunoassays

BNP precursor (proBNP) is a polypeptide molecule containing 108 amino acid residues. ProBNP can be detected in patients' blood at concentrations comparable with the concentration of circulating BNP and NT-proBNP. Thus proBNP measurements could be of clinical value in cardiovascular disease diagnosis, prognosis, and treatment monitoring. MAbs specific to peptides 5-12 and 13-27 of SEQ ID NO:1 (proBNP) recognize circulating proBNP and could be used for the development of quantitative proBNP assays in pairs with BNP-specific MAbs 50E1 and 2G9 (e.g., Hytest Ltd. cat. no. 4BNP2). Preferred MAb pairs (capture-detection) are:

### 50E1-16F3

### 50E1-18H5

### 7B5-2G9.

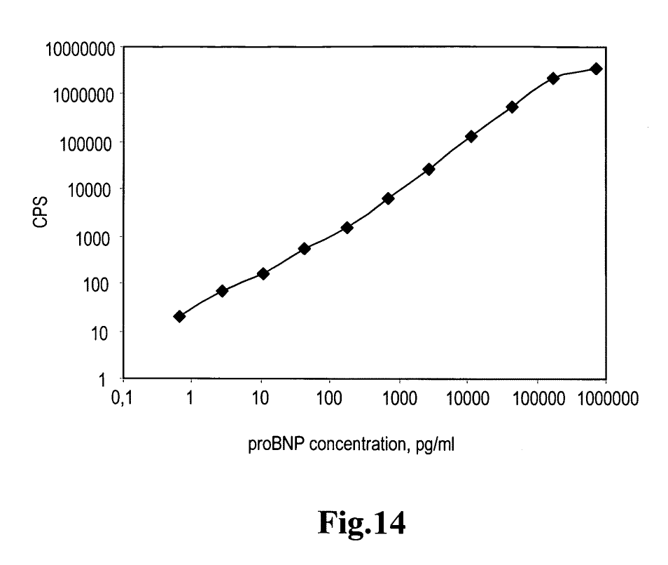

Each of these pairs of anti-proBNP monoclonal antibodies demonstrates high sensitivity, good kinetics and effective recognition of antigen in patients' blood (FIG. 14).

4. NT-proBNP and proBNP Immunodetection by Western Blotting

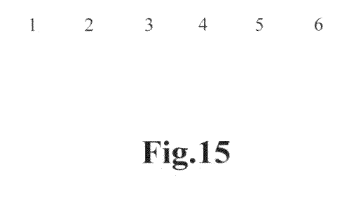

The above-identified monoclonal antibodies from Hytest Ltd. recognize human NT-proBNP and proBNP in Western blotting studies after antigen transfer onto nitrocellulose membrane, as revealed in FIG. 15.

The human recombinant NT-proBNP and proBNP used in the experiments described herein exhibit the following properties:

| | |
|---|---|
| Purity: | >95% according to SDS-PAGE |
| Application: | NT-proBNP and proBNP immunoassay calibrators |
| | and standards, immunoblotting. |

Human recombinant NT-proBNP (rec NT-proBNP; a.a.r. 1-76; SEQ ID NO:3) and human recombinant proBNP (rec proBNP; a.a.r. 1-108; SEQ ID NO:1) are expressed in E. coli. Both polypeptides have the same sequence as natural proteins with a single amino acid difference, an additional Met residue from the N-terminus of the molecule. Antigens are recognized by poly- and monoclonal antibodies specific to different parts of NT-proBNP. ProBNP is also recognized by antibodies specific to BNP.

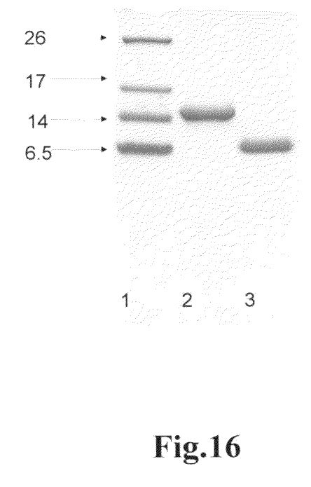

Both rec NT-proBNP and rec proBNP (Hytest Ltd.) are highly purified peptides. Purity is more than 95% according to SDS-PAGE (FIG. 16) and HPLC studies. The antigens could be used as calibrators or as standards in NT-proBNP and proBNP assays. In BNP clinical immunoassays, proBNP or a fragment thereof is preferred as a standard or calibrator. Also preferred as a calibrator in BNP immunoassays is a modified form of proBNP, as defined hereinabove.

Although a variety of BNP immunoassays are useful in measuring BNP immunoreactivity in a variety of samples, superior results are obtained from immunoassays in which a stable standard or calibrator peptide, such as proBNP, is used. The stability studies described below show that recombinant proBNP demonstrated significantly higher stability than synthetic BNP-32. In addition, among recombinant proBNPs, the glycosylated forms of proBNPs expressed in eukaryotic cell lines are significantly more stable than non-glycosylated proBNPs expressed in bacterial cells.

A preferred proBNP for the purposes of the invention as a standard or calibrator preparation for use in a method for detecting BNP immunoreactivity in a sample is glycosylated proBNP, which may be endogenous or, alternatively, recombinant proBNP expressed in a eukaryotic cell line.

The amino acid sequence of proBNP is provided in SEQ ID NO:1, the amino acid sequence of the whole preproBNP in SEQ ID NO:2, the amino acid sequence of NT-proBNP in SEQ ID NO:3, and the amino acid sequence of the mature BNP in SEQ ID NO:4.

A “sample of a patient” or “patient sample” for the purposes of the invention is usually a body fluid sample obtained form a patient. It is, for example, a serum or plasma fraction obtained from a blood sample drawn form a patient.

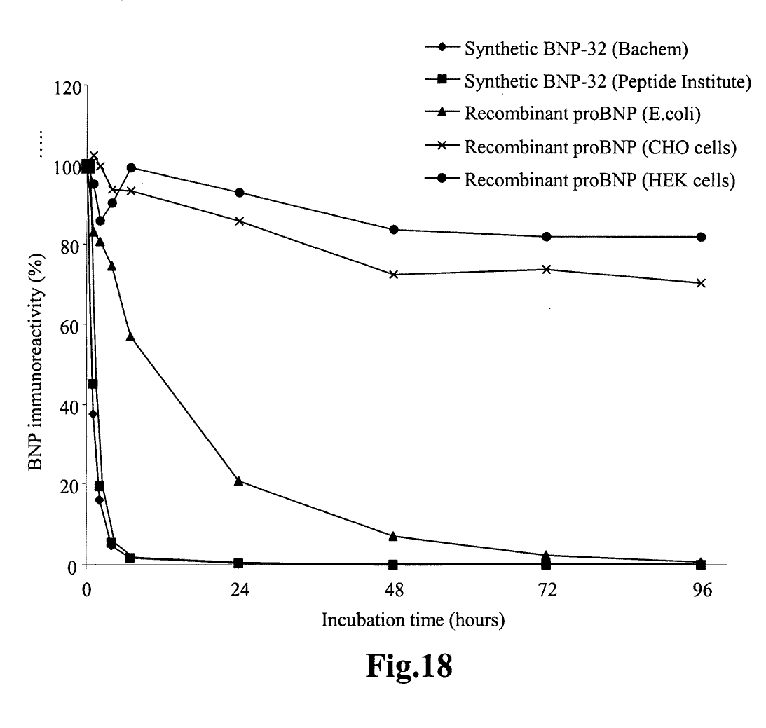

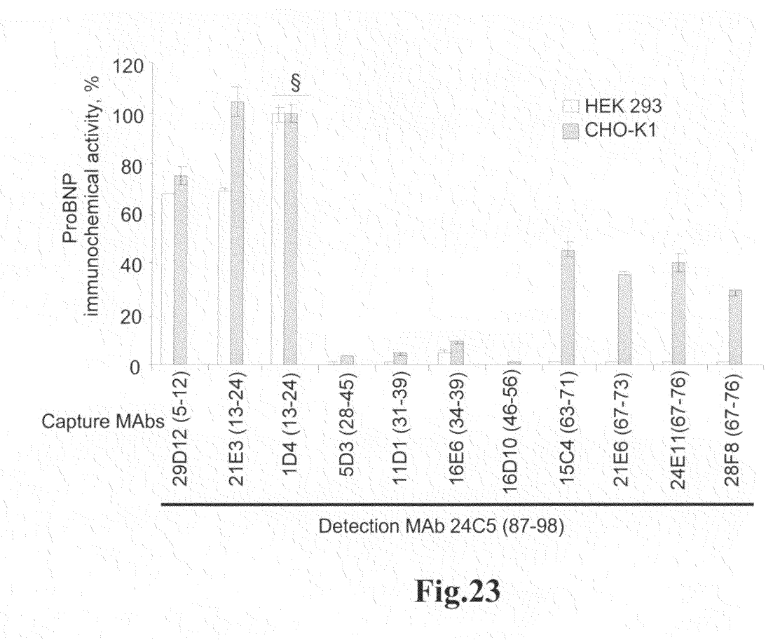

The appended FIG. 18 (see also Example 14) shows that glycosylated forms of the recombinant proBNP expressed in two different eukaryotic cells (HEK 293 and CHO) display different stabilities, although both forms are glycosylated. In our latest studies it was demonstrated that antibodies specific to region 63-76 cannot recognize proBNP expressed in HEK 293 cells, but can recognize about 40% of proBNP expressed in CHO-K1 cells (FIG. 23). In recombinant proBNP expressed in CHO-K1 cells about 60% of Thr71 residues are glycosylated, whereas in proBNP expressed in HEK 293 cells Thr71 is glycosylated almost completely. Complete glycosylation of Thr71 residue in proBNP molecule expressed in HEK 293 cells makes it much more stable in comparison with proBNP expressed in CHO-K1 cells. This made us conclude that not glycosylation in general, but glycosylation in one particular amino acid—Thr71—is crucial for proBNP stability.

Glycosylation of Thr71 in proBNP molecule makes it impossible to use antibodies specific to the epitopes which comprise this amino acid residue in proBNP immunoassays. For precise immunodetection of proBNP in patient's blood one antibody in sandwich immunoassay should be specific to the BNP portion (amino acid residues 77-108) of proBNP molecule, whereas another one should be specific to the N-terminal part of proBNP (amino acid residues 1-34), which is not susceptible to glycosylation.

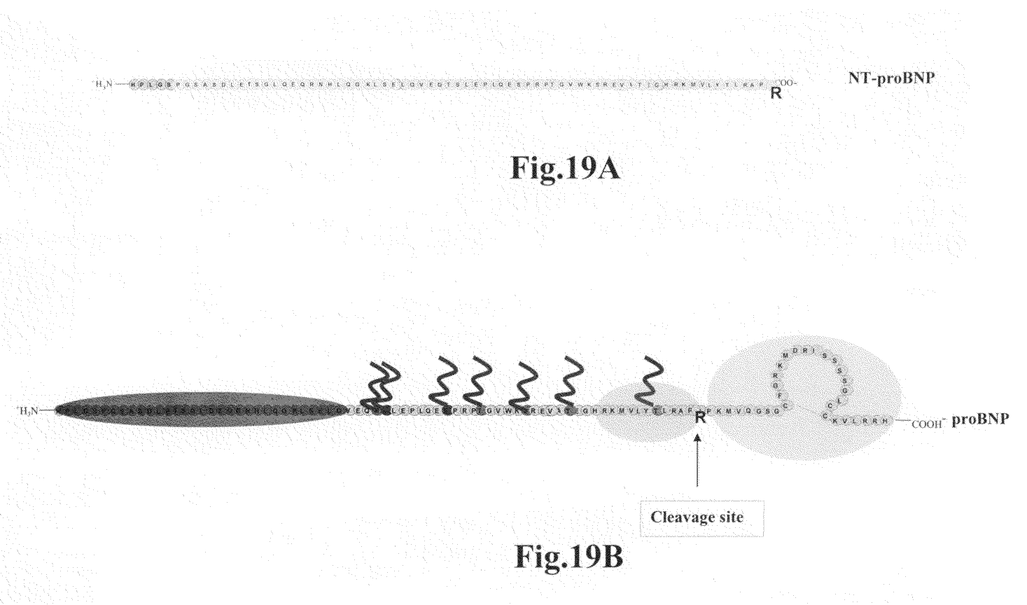

In contrast, the region 61 to 76 aar in NT-proBNP is recognized by MAbs specific to this region. According to the recent findings of the present inventors the central part (amino acids from about 31 to about 60) of NT-proBNP circulating in human blood is glycosylated (FIG. 19A, yellow oval), whereas N- and C-terminal portions (FIG. 19A, blue ovals; corresponding to amino acids from about 6 to about 29 and from about 61 to about 76 of NT-proBNP, respectively) do not contain glycosylated sites. Consequently, antibodies specific to N- and C-terminal parts of NT-proBNP could be used for endogenous NT-proBNP immunodetection.

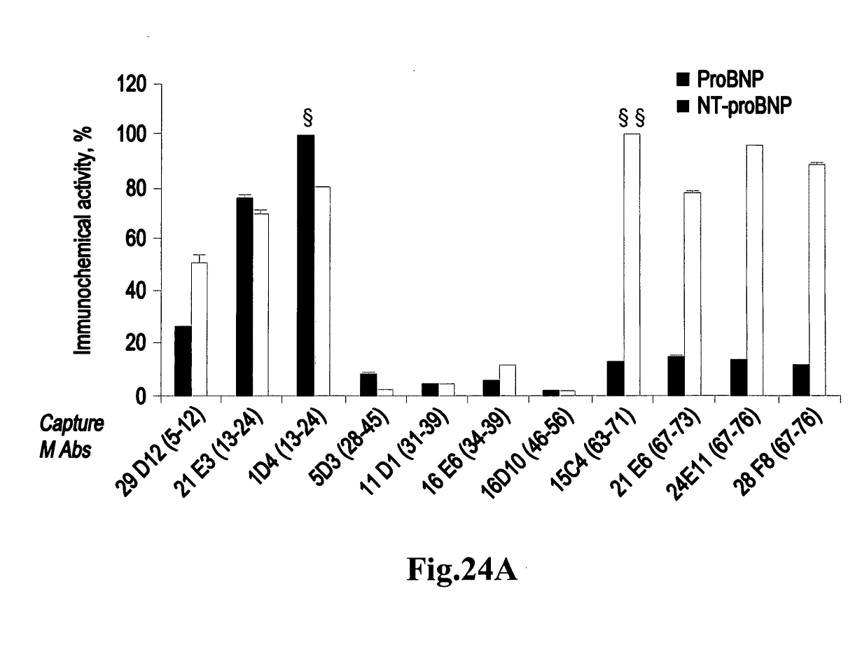

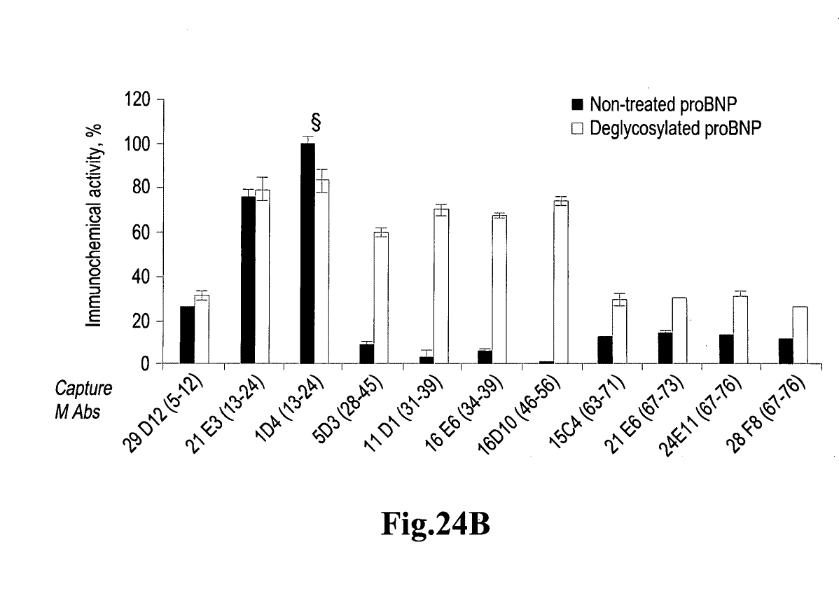

As demonstrated in FIG. 24, the fragment of endogenous proBNP from 61 to 76 aar, in contrast to the same region on NT-proBNP, is not recognized by region-specific MAbs. We compared the immunoreactivity profiles of proBNP and NT-proBNP from HF patients' plasma using MAbs specific to different regions of NT-proBNP molecule. The profile of immunochemical activity for both molecules was similar with the exception for the region 61-76 located close to the cleavage site 76R↓S77 (FIG. 24A). The region 61-76 of endogenous proBNP was inaccessible to specific MAbs, whereas the same antibodies recognized endogenous NT-proBNP with high efficiency. Since proBNP was shown to be an O-glycoprotein (Schellenberger et al, 2006), we hypothesized that glycosylation could be the reason why antibodies are unable to recognize the region 61-76 of endogenous proBNP. To examine this hypothesis, proBNP extracted from HF patients' plasma was treated with mixture of O-specific glycosidases. Deglycosylation resulted in 2-2.3-fold increase of immunoreactivity of proBNP in the assays using MAbs specific to the region 61-76 (FIG. 24B). So it was concluded that region located close to the cleavage site is glycosylated in endogenous proBNP in comparison with NT-proBNP.

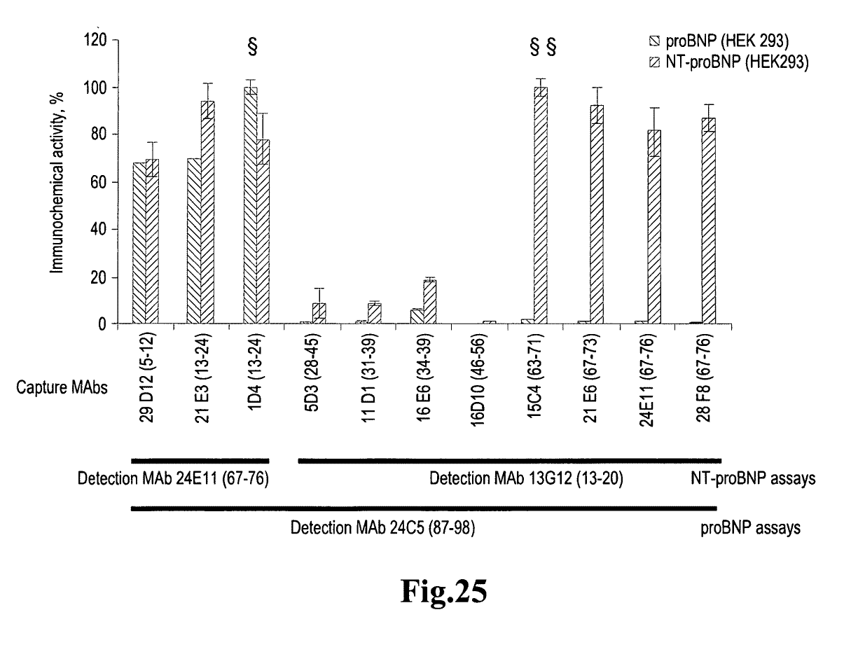

When proBNP was expressed in HEK 293 cells by means of transient transfection, both proBNP and products of its processing—BNP and NT-proBNP—were detected in conditioned media. We compared the immunoreactivity profiles of recombinant proBNP and NT-proBNP using MAbs specific to different regions of proBNP molecule. Similarly to endogenous peptides, in recombinant NT-proBNP C-terminal part of the molecule was not glycosylated (the region was accessible for region-specific MAbs), whereas the same region of proBNP molecule was heavily glycosylated (the region was inaccessible to region-specific MAbs) (FIG. 25).

The level of glycosylation of Thr and Ser residues in the proBNP molecule is variable in population, and is thus different in the blood of each individual and most likely it is dependent on the biochemical status of the donor. The level of Thr71 glycosylation correlates with the level of glycosylation of other Thr and Ser residues from the central part of the molecule. The higher is the level of glycosylation the lower is the part of the synthesized protein which is able to be converted into NT-proBNP and BNP, i.e. the lower portion of synthesized proBNP will be converted into the physiologically active peptide BNP. This inability to convert the prohormone to the hormone itself can result in serious pathophysiological outcomes. The concentration ratios of the different forms, e.g. proBNP/BNP, proBNP/NT-proBNP or NT-proBNP/proBNP, in patient's blood may be a very important diagnostic and prognostic parameter.

The ratio of NT-proBNP/proBNP concentrations (or vice-versa) in human blood could be of great clinical value because it probably can explain or predict development of pathology in time (for instance heart failure, HF) associated with high (or low) level of proBNP glycosylation in a patient's heart.

### EXPERIMENTAL

### Example 1

### Preparation and Characterization of Monoclonal Antibodies (Mabs), Specific for Human NT-proBNP Molecule

Synthetic peptides corresponding to sequences 1-24, 13-27, 28-45, 46-60 and 61-76 of human NT-proBNP molecule (HyTest Ltd., Finland) were conjugated to the bovine serum albumin (BSA) and were used for immunization of mice. Conjugation of small peptides with the carrier protein molecule allowed enhancing the immune response of the animals.

Female Balb/c mice, aged between 6-12 weeks were used for immunization.

The hybridoma cell lines producing monoclonal antibodies (MAbs), specific to NT-proBNP molecule were obtained after hybridization of mouse spleen cells with myeloma SP2/0 cells.

Culture supernatants were tested for reactivity to the whole recombinant NT-proBNP molecule (expressed in E. coli) and eighty-five positive cultures were selected for further work. Among those, 14 produced antibodies specific to region 1-24, 24 to region 13-27, 19 to region 28-45, 13 to region 46-60 and 15 to region 61-76. Selected cultures were subcloned twice by limiting dilution, expanded and frozen.

The ascitic fluid containing monoclonal antibodies was produced in Balb/C mice.

The antibodies were isolated from the ascitic fluid by Protein-A Sepharose (GE Healthcare) affinity chromatography.

The isotypes of the purified antibodies were determined by Monoclonal Antibody Isotyping Kit (Pierce). All MAbs were specified as IgG.

### Example 2

### Epitope Analysis

Precise epitope mapping of all newly generated antibodies was performed using a library of synthetic peptides 1-12, 5-20, 1-24, 13-27, 28-45, 31-39, 34-42, 37-45, 48-56, 50-58, 52-60, 46-60, 63-71, 65-73, 67-76 and 61-76, containing overlapping sequences. Synthetic peptides were conjugated with a carrier protein (ovalbumine). The plates were coated with peptide conjugates in concentration of 1 μg/ml (100 μl per well). After washing, monoclonal antibodies, reconstituted in PBST, were added into the wells. After 30-minute incubation at room temperature the plates were washed and HRP-conjugated rabbit anti-mouse Fc-specific polyclonal antibodies were added to each well. After 30-minute incubation the plates were washed with PBST, and color development was achieved with the o-phenylenediamine substrate system. Absorbance was measured at 492 nm using Victor 1420 Multilabel Counter. Four groups of antibodies with epitopes located in regions 1-12, 5-12, 5-20 and 13-24 were discriminated among MAbs specific to the region 1-24. Two groups of antibodies with epitopes 13-20, 13-24 and 31-39, 34-39 were discriminated among antibodies specific to regions 13-27 and 28-45, respectively. Four groups of antibodies with epitopes 46-56, 48-56, 46-60 and 52-58 were discriminated among antibodies specific to peptide 46-60 and three groups, 63-71, 67-73 and 67-76, among the antibodies specific to peptide 61-76.

### Example 3

### Development of Sandwich Immunofluoroassays (IFA) for Quantitative Measurement of Human NT-proBNP

According to the invention sandwich-type immunofluoroassays were established for the quantification of NT-proBNP in human blood. Such assay is based on the binding of the antigen to the monoclonal antibody adsorbed on the plate surface thus forming first order immune complex, and on the detection of the first order immune complex by another monoclonal antibody labeled with stable europium (III) chelate.

### Example 4

### Conjugation of Antibodies with Stable Europium Chelate

Antibodies were preliminarily transferred into 0.9% water solution of NaCl using gel filtration on Sephadex G25 columns (NAP-5). Antibody labeling with stable europium (III) chelate of 2,2′,2″,2′″-[[4-[(4-isothiocyanatophenyl)ethynyl]pyridine-2,6-diyl]bis(methylenenitrilo)]tetrakis(acetic acid) was conducted by incubation overnight at +4° C. in 50 mmol/L Na-carbonate buffer pH 9.8 containing 200-fold molar excess of europium (III) chelate. Labeled antibodies were separated from the unreacted chelate by gel filtration on Sephadex G25 columns (NAP-5) in a buffer containing 0.01 mol/L of Tris-HCl pH 7.8, 0.15 mol/L of NaCl and 0.1% NaN₃.

### Example 5

### Patients and Blood Samples

Diagnosis of patients with HF was based on symptoms: dyspnea, orthopnea, lung rates and leg edema, and confirmed by echocardiography studies and X-ray examination. The preliminary diagnosis was made by cardiologist and further confirmed by HF expert. Blood samples were collected from patients with left ventricular ejection fractions less than 30% and left ventricle end-systolic volume more than 90 mL. Venous blood was collected into EDTA-containing Vacuette tubes (Greiner Bio-One) and centrifuged at 3000 g (15 minutes, +4° C.). Serum samples were obtained from blood collected in plastic tubes, incubated for 30 min at room temperature, and centrifuged at 5000 g (30 min, +20° C.). Plasma and serum samples were stored at −70° C. prior to use. For MAb testing, pooled serum (39 patients with severe HF) or pooled plasma (10 HF patients) was used as a source of endogenous antigens. As a negative (non-HF) control pooled serum or plasma from 10 healthy donors was used.

### Example 6

### Sandwich IFA

All generated MAbs were tested in two-site MAb combinations (capture and detection) with recombinant NT-proBNP and with pooled serum or plasma from HF patients as a source of endogenous antigens. The capture antibodies in concentration of 10 μg/ml were placed into EIA plates (100 μl per well) and incubated in a phosphate saline buffer for 30 minutes at room temperature and gentle shaking. After twofold washing of the titration plates with a buffer containing 0.01 mol/L Tris-HCl pH 7.8, 0.15 mol/L NaCl, 0.025% Tween 20 and 0.05% NaN₃ (buffer A), the mixture of antigen (recombinant NT-proBNP, reconstituted in pooled normal human plasma or endogenous antigen from HF plasma or serum, 50 μl) and detection antibodies (4 μg/ml, 50 μl), dissolved in buffer containing 0.05 mol/L Tris-HCl pH 7.7, 0.9% NaCl, 0.01% Tween 20, 0.5% BSA and 0.05% NaN₃ (buffer B) were added to the wells. The plates were incubated for 30 minutes at room temperature and shaking gently, and washed six times with buffer A. After addition of the enhancing solution (1.75 mol/L NaSCN, 1 mol/L NaCl, 50 ml/L glycerol, 200 ml/L 1-propanol, 0.005 mol/L Na₂CO₃, 0.05 mol/L glycine-NaOH, pH 10.0), the mixture was incubated for 3 minutes at the same conditions. The fluorescence was measured on a Victor 1420 Multilabel Counter.

### Example 7

### Blood Sample Testing in Different Immunoassays

All MAbs with remote epitopes being utilized in 2-site combinations (sandwich immunoassays) were able to recognize recombinant NT-proBNP and proBNP with a low detection limit (10-100 ng/L). At the same time, only few MAb combinations were able to recognize antigen from serum and plasma of HF patients. Only two-site combinations utilizing one MAb specific to region 13-27 and another MAb specific to region 61-76 recognized the endogenous antigen with high sensitivity.

None of the 13 MAbs specific to region 46-60 were able to recognize the endogenous antigen being used in 2-site combinations with any other antibody. When MAbs specific to the very N-terminal region or to region 28-45 were tested in pairs with antibodies recognizing sequence 61-76, the ratio of signals from the endogenous to recombinant peptide was significantly lower than in the assays utilizing MAbs specific to epitope 13-27. Only MAb 29D12, which is specific to peptide 5-12, elicited high signals upon interaction with either serum or plasma samples being tested in pairs with MAbs specific to peptide 67-76.

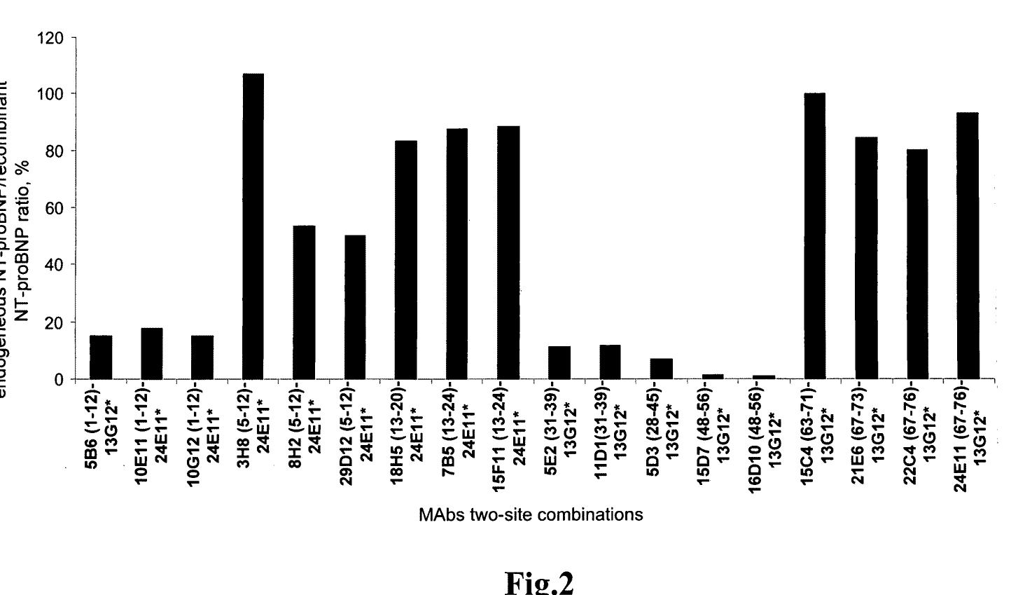

Results of testing of recombinant and endogenous (pooled plasma of heart failure subjects) forms of NT-proBNP in sandwich immunoassays, utilizing antibodies specific to different regions of NT-proBNP molecule are presented in FIG. 2.

The assay 15C4₆₃₋₇₁-13G12₁₃₋₂₀ was selected and used in further studies because it was able to recognize recombinant and native antigens with the same efficiency.

Consequently, we demonstrated that MAbs specific to the central part (region 28-60) of NT-proBNP molecule are almost unable to recognize the antigen in human blood.

### Example 8

### Characterization of 15C4-13G12 Immunoassay

Sandwich immunofluorometric assay utilizing MAb 15C4-13G12 was performed as described in Example 6.

The specificities of antibodies used in the assay were confirmed by Western blotting analysis (data not shown). Both of the antibodies detected recombinant proBNP and NT-proBNP expressed in E. coli.

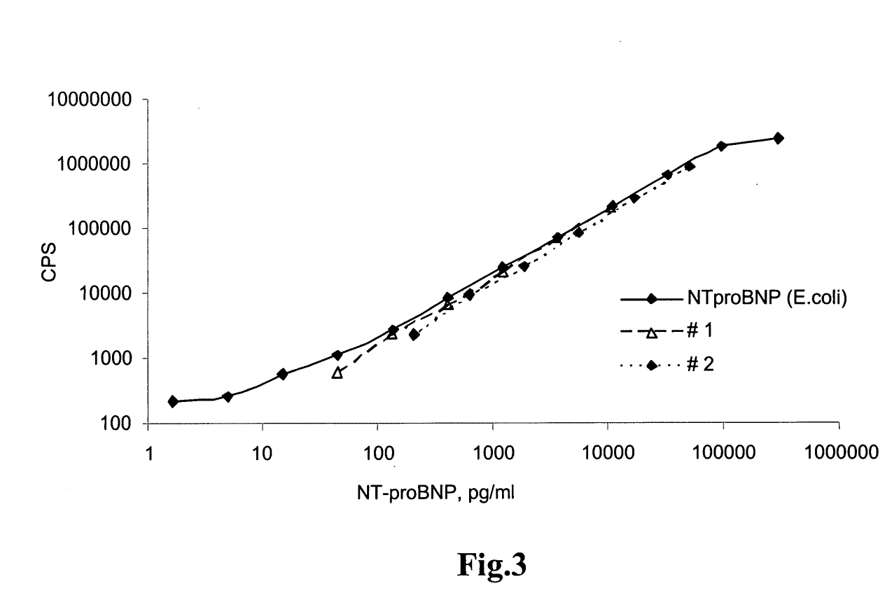

Human recombinant NT-proBNP expressed in E. coli was reconstituted in normal human plasma and was used as a calibrator for sandwich IFA. The detection limit was defined as a concentration of the calibrator (measured 20 times in a single run) producing a signal 2 SD above the mean for a matrix that is free of analyte. Typical calibration curve for recombinant NT-proBNP and serial dilutions of human plasma samples are shown in FIG. 3. The detection limit was 10 ng/L, immunoassay was linear in the range of 15-100 000 ng/L.

### Example 9

### Gel Filtration Studies of Recombinant and Endogenous NT-proBNP

Seidler et al. (1999) demonstrated that in gel filtration studies (in non-denaturing conditions) NT-proBNP and proBNP have anomalous mobility and their apparent molecular weights are about 30-40 kDa. The authors suggested that in human blood both proteins are presented in homo-oligomeric forms. In our studies we carried out gel filtration studies using HF patients' plasma as a source of endogenous NT-proBNP.

Individual patient plasma samples were centrifuged at 10 000 g for 10 minutes and supernatants (150 μl) were applied onto Superdex 75 10/300 GL gel filtration column equilibrated with the buffer containing 0.1 mol/L sodium phosphate, pH 7.4, 0.3 mol/L NaCl and 0.005 mol/L EDTA. Proteins were eluted at a flow rate 0.8 ml/min and fractions with a volume of 0.7 ml were collected. Subsequently, the NT-proBNP immunological activity was measured in 15C4₆₃₋₇₁-13G12₁₃₋₂₀ sandwich immunoassay.

The column was calibrated using a set of standard proteins (GE Healthcare): albumin (Mr 67000 Da), ovalbumin (Mr 43000 Da), chymotrypsinogen (Mr 25000 Da), ribonuclease A (Mr 13700 Da) and aprotinin (Mr 6517.5 Da, from Sigma). Recombinant NT-proBNP was reconstituted in pooled plasma from healthy donors before loading onto the Superdex 75 column.

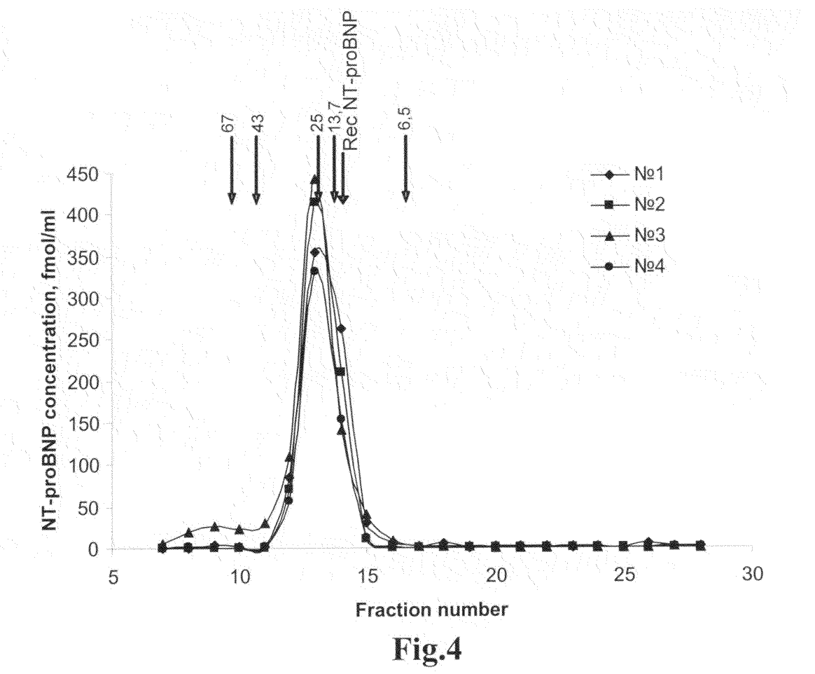

Fraction analysis by the NT-proBNP assay revealed one peak of immunoreactivity (about 30 kDa) in human plasma samples (FIG. 4). The position of the NT-proBNP activity maximum did not coincide with those established for recombinant NT-proBNP (8.46 kDa) and recombinant (non-glycosylated) proBNP (11.9 kDa). We thus observed that endogenous NT-proBNP in GF has significantly higher apparent molecular weight than the recombinant protein. The observed differences in apparent molecular weights of endogenous and recombinant proteins could be explained by posttranslational modifications (in the central part of the molecule) of endogenous NT-proBNP. The existence of such modifications could also explain the fact that antibodies specific to the central region of the molecule are not able to recognize the endogenous antigen.

### Example 10

### Western Blotting Studies of Endogenous NT-proBNP

NT-proBNP was purified from pooled HF patients' plasma by means of affinity chromatography. To obtain the affinity matrix, a mixture of antibodies, specific to different regions of NT-proBNP molecule (15C4, 24E11, 18H5, 15F11), was immobilized on the BrCN-activated Sepharose CL-4B (GE Healthcare) according to the standard protocol. In the above-described experiments (see Example 8) it was demonstrated that the above-mentioned MAbs were able to recognize the endogenous antigen with high efficiency. Affinity matrix with immobilized antibodies was washed with 0.1 mol/L of glycine, pH 2.0 and then equilibrated with 0.02 mol/L Tris-HCl, pH 7.5, containing 0.15 mol/L of NaCl. Pooled plasma of HF patients was loaded onto anti-NT-proBNP-Sepharose with the flow rate of 1 ml/min at +4° C. Peptides were eluted with a water solution containing 0.1 mol/L of HCl. The eluate was neutralized by 2 mol/L of Tris-HCl. Recovery after affinity chromatography was 88%.

The eluate was then loaded onto Sepharose CL-4B with immobilized MAbs that do not interact with NT-proBNP (negative chromatography) to remove proteins that bind nonspecifically to the affinity matrix.

Subsequently, NT-proBNP was concentrated by a second round of affinity chromatography. The solution containing NT-proBNP was loaded onto anti-NT-proBNP-Sepharose (10-fold molar excess of antibodies regarding to the NT-proBNP concentration) with the flow rate of 1 ml/min at +4° C. The peptides were eluted by 0.1 mol/L of HCl, lyophilized, and stored before use under −70° C.

The proBNP contamination of the NT-proBNP preparation was determined by proBNP-specific immunoassay and was found to be less than 9% from total amount of NT-proBNP.

Samples of endogenous NT-proBNP as well as recombinant NT-proBNP and proBNP were boiled for 5 minutes in 0.25 mol/L Tris-HCl pH 6.8, 10 ml/L 2-mercaptoethanol, 20 g/L sodium dodecyl sulfate and 100 ml/L glycerol and loaded onto gel for protein separation by means of Tricine electrophoresis as described by Schagger and von Jagow (1987). The electrophoresis was performed at constant voltage (150 V) under +4° C. for 4 hours, using 16.5% T, 3% C separating gel. 200 ng of NT-proBNP (concentration was determined by 15C4₆₃₋₇₁-13G12₁₃₋₂₀ immunoassay) extracted from pooled HF patients' plasma, or 50 ng of recombinant NT-proBNP (proBNP) were loaded per track.

After the electrophoresis the peptides were transferred onto nitrocellulose membrane (Trans-Blot® Transfer membrane, 0.2 μm, BioRad). Transfer was performed at constant voltage (100 V) and lasted for 40 minutes. Nonspecific binding was blocked by incubation of the membrane in the 10% solution of non-fat dry milk in PBST. Immunochemical staining of the peptides with NT-proBNP-specific MAbs, conjugated with horseradish peroxidase was performed during 12 hours at +4° C. in the 10% solution of non-fat dry milk in PBST. Immune complexes were visualized by incubation in substrate solution, containing diaminobenzidine and nickel chloride.

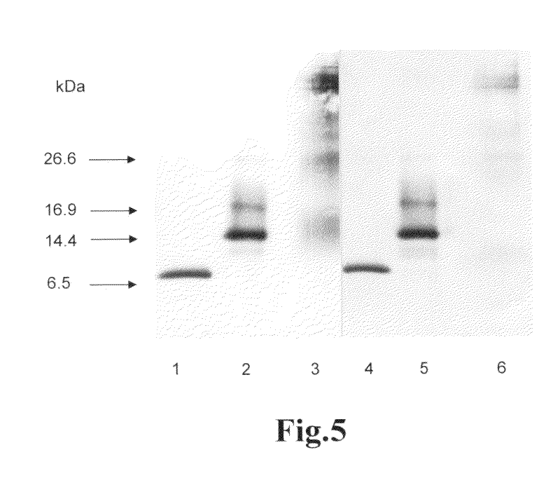

It was shown that in the tracks containing endogenous protein none of the tested monoclonal antibodies recognizes any protein band with the same molecular mass as recombinant NT-proBNP (FIG. 5). When tested by MAb 15F11 (epitope 13-24) an immunoreactive protein was detected in several diffused zones in the area corresponding to the proteins with molecular masses of 15 kDa and higher. In contrast to MAb 15F11, endogenous NT-proBNP was not stained by MAb 11D1 specific to the region aar 31-39. These data are in agreement with the results of IFA described in Example 7, where 11D1 scarcely detected NT-proBNP in human plasma.

The existence of different diffused NT-proBNP zones stained by MAb 15F11 in Western blotting studies could be explained by glycosylation of NT-proBNP molecules circulating in human blood.

### Example 11

### Testing of Deglycosylated Endogenous NT-proBNP in Sandwich IFA

NT-proBNP extracted from pooled human plasma was treated by deglycosylation enzymes O-glycosidase (S. pneumoniae) and sialidase (A. ureafaciens) (QA-Bio, USA). Treatment was performed in a buffer containing 0.075 mol/L of sodium phosphate, pH 5.0 for 1 hour at +37° C. Water solution, containing 0.075 mol/L of sodium phosphate, pH 5.0 without enzymes was added to the studied peptides as a negative control.

After deglycosylation endogenous NT-proBNP was tested in immunoassays utilizing MAbs specific to different parts of the molecule as described in Example 7.

Immunological activities measured in assays utilizing MAbs specific to the central part of the molecule increased significantly after deglycosylation (FIG. 6). In the assays utilizing MAbs 5D3₂₈₋₄₅, 11D1₃₁₋₃₉, 5E2₃₁₋₃₉, 16E6₃₄₋₃₉ the signal increased 7.5-10 fold. For MAbs 15D7₄₈₋₅₆, and 16D10₄₈₋₅₆ specific to the region 46-56 of NT-proBNP the immunological activity increased about 50 fold. Results in FIG. 6 are presented as a signal ratio of endogenous (treated or non-treated)/recombinant NT-proBNP (%), where the ratio of signals endogenous deglycosylated/recombinant in the assay utilizing MAbs 15C4-13G12 was taken as 100%.

Consequently, we have shown that the central portion of the NT-proBNP molecule is glycosylated, and polysaccharide residues prevent antibodies from interacting with the endogenous antigen. Since antibodies specific to the central part of the NT-proBNP molecule are unable to recognize endogenous antigen, MAbs specific to the other regions not affected by glycosylation should be used in NT-proBNP and proBNP assays.

### Example 12

### Gel-Filtration Studies of Endogenous NT-proBNP Before and After Deglycosylation

NT-proBNP treated and non-treated with the O-glycosidase and sialidase was studied by gel-filtration (GF) method. NT-proBNP, extracted from pooled HF patients' plasma, was treated with enzymes and the immunological activity was thereafter determined in fractions in two immunoassays 15C4-13G12 and 11D1-13G12. According to the data presented in Example 11, assay 15C4-13G12 is not sensitive to glycosylation, whereas 11D1-13G12 assay can recognize endogenous NT-proBNP only after removal of carbohydrate moieties.

The samples of a) endogenous NT-proBNP, extracted from pooled HF patients' plasma, b) endogenous NT-proBNP, extracted from pooled BF patients' plasma after deglycosylation, c) recombinant NT-proBNP and d) recombinant proBNP in the same concentrations (330 ng/ml) were reconstituted in 150 μl of 0.1 mol/L sodium phosphate, pH 7.4, containing 0.7 mol/L of NaCl, 0.005 mol/L of EDTA and 5 g/L of bovine serum albumin. The samples were applied onto Superdex 75 10/300 GL gel filtration column equilibrated with 0.1 mol/L of sodium phosphate, pH 7.4, containing 0.7 mol/L of NaCl and 0.005 mol/L of EDTA. Proteins were eluted at a flow rate of 0.7 ml/min and fractions with a volume of 0.5 ml were collected. The NT-proBNP immunological activities in the fractions were measured by sandwich immunoassay 15C4-13G12, utilizing monoclonal antibodies not sensitive to glycosylation and 11D1-13G12 assay that does not interact with glycosylated endogenous NT-proBNP.

The column was calibrated using the set of standard proteins: albumin (Mr 67000 Da), ovalbumin (Mr 43000 Da), chymotrypsinogen (Mr 25000 Da), ribonuclease A (Mr 13700 Da) and aprotinin (Mr 6517.5 Da). Recombinant NT-proBNP and proBNP were reconstituted in pooled plasma From healthy donors before loading onto the Superdex 75 column.

Being measured in the 15C4-13G12 immunoassay endogenous NT-proBNP revealed two peaks of immunoreactivity (FIG. 7), the major one with molecular weight of about 28 kDa and the minor one with molecular weight of about 51 kDa. After deglycosylation we observed shift of both peaks of immunological activity towards the proteins with lower molecular masses. The major peak corresponded to the proteins with molecular masses of about 18 kDa and the minor peak to the proteins with molecular masses of about 40 kDa.

Being measured in the 11D1-13G12 immunoassay (utilizes antibody 11D1, sensitive to glycosylation), endogenous NT-proBNP gave almost no response, whereas significant response was observed in the case of deglycosylated protein. Profile of immunoreactivity was very similar to the profile, measured by 15C4-13G12 immunoassay.

### Example 13

### Immunochemical Staining of Affinity-Purified Endogenous NT-proBNP (Before and After Deglycosylation) by NT-proBNP-Specific Monoclonal Antibodies in Western Blotting

Electrophoresis and Western blotting procedure were performed in the same way as described in Example 10.

In this case MAb 15F11 (epitope 13-24) was used for the protein visualization in the sample before deglycosylation. The major immunological activity was detected as a diffused zone in the area corresponding to the proteins with molecular masses of about 15 kDa and higher. In contrast to MAb 15F11, endogenous NT-proBNP was not stained by MAb 11D1 specific to the region aar 31-39 of NT-proBNP molecule. After deglycosylation both of the antibodies were able to detect a peptide with apparent molecular mass about 13 kDa. This band was still a little above the recombinant NT-proBNP band, which could be explained by the fact that deglycosylation was not complete.

We thus conclude that the endogenous protein cannot be detected in Western blotting experiments by antibodies specific to the central region of NT-proBNP molecule, whereas it becomes “visible” by such antibodies after deglycosylation.

### Example 14

### Stability Studies

Antigens used in the studies. The antigens used in the stability studies included synthetic BNP-32 (Bachem, Bubendorf, Switzerland, cat. no. H-9060.0500), synthetic BNP-32 (Peptide Institute, Osaka, Japan, cat. no. 4212-v), recombinant proBNP (expressed in E. coli, non-glycosylated, from Hytest Ltd., Turku, Finland, 8PRO8), recombinant proBNP (expressed in eukaryotic CHO (Chinese hamster) cells and, thus, glycosylated, as described herein), and recombinant proBNP (expressed in eukaryotic HEK (human) cells and, thus, glycosylated, as described herein).

Antigen solutions. An antigen matrix was prepared by pooling human citrate plasmas collected from healthy donors. Plasma was obtained from 15 healthy volunteers by vein puncture and mixed with 0.11 M sodium citrate tribasic in a ratio of 4:1. The crude serum:citrate was centrifuged for 30 minutes at 2000 rpm. Plasma was decanted, pooled and stored at −70° C. until use. Antigen concentrations used in the studies were 6 n/ml for recombinant proBNP expressed in CHO cells, 20 ng/ml for synthetic BNPs, 20 ng/ml for recombinant proBNP expressed in E. coli and 20 ng/ml for recombinant proBNP expressed in HEK cells. 0.1% sodium azide was added to plasma as a preservative to prevent bacterial growth.

Incubation at 4° C. and 25° C. Antigen solutions in human plasma were incubated for different time periods (up to 96 hours) at 4° C. and 25° C. Aliquots were frozen and stored at −70° C. until BNP immunoreactivity was measured.

Assays for BNP immunoreactivity. BNP immunoreactivity in the samples was determined by sandwich immunofluorometric assay utilizing two BNP-specific monoclonal antibodies (MAbs). A first BNP-specific monoclonal antibody was coating monoclonal antibody, or MAb, 50E1 (Hytest Ltd. cat. no. 4BNP2), which specifically recognized and bound to peptide 26-32 of the BNP sequence. A second BNP-specific monoclonal antibody was detection monoclonal antibody, or MAb, 24C5 (Hytest Ltd. cat. no. 4BNP2). Also suitable for use in such assays are a variety of antibodies specific to BNP, such as additional antibodies provided by Hytest Ltd. under cat. no. 4BNP2, i.e., 26E2, 2G9, 50B7, 57H3, 41A6 and 43B12 as well as other monoclonal or polyclonal antibodies of various isotypes, and fragments or derivatives that retain the capacity to specifically bind to BNP.

MAb 24C5 specifically recognized and bound to peptide 11-22 of the BNP sequence and was conjugated to stable Eu³⁺ chelate using conventional techniques. Such an assay is able to detect not only BNP but also proBNP. Consequently, it can be used for the quantification of whole BNP immunoreactivity, comprising both BNP and proBNP.

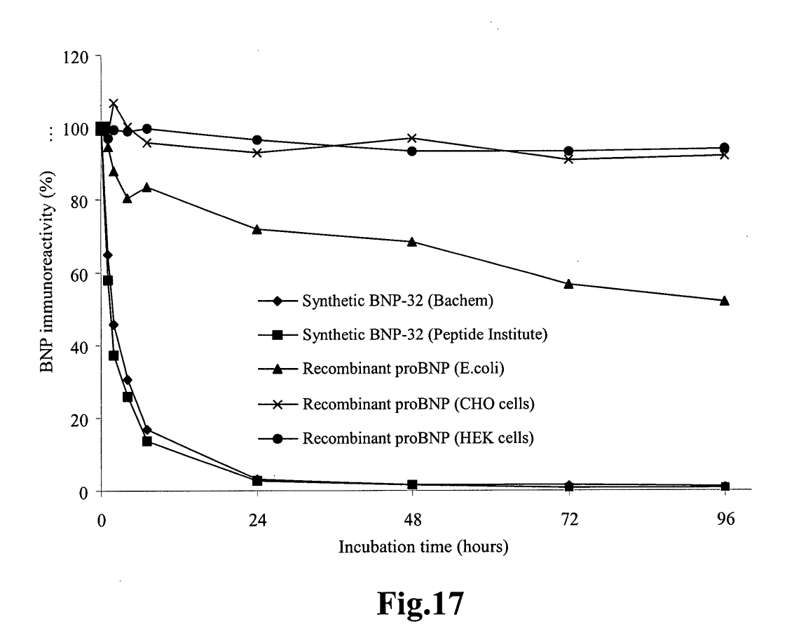

The results of the stability studies are presented in FIG. 17 and FIG. 18. Synthetic BNP-32 demonstrated the worst stability among all of the tested protein forms displaying BNP immunoreactivity. 3% and less of initial immunoreactivity were observed in the samples after 24 hours of incubation at 4° C. and less than 0.5% after 24 hours of incubation at 25° C. Almost no immunoreactivity was detected after 96 hours of incubation at both temperatures.

Recombinant non-glycosylated proBNP, expressed in E. coli, demonstrated intermediate stability. More than 50% of initial proBNP immunoreactivity was still detected after 96 hours of incubation at 4° C. At 25° C. recombinant proBNP, expressed in E. coli, demonstrated significantly lower stability than after incubation at 4° C., i.e., less than 1% of initial proBNP immunoreactivity was detected after 96 hours of incubation at 25° C.

Recombinant glycosylated forms of proBNP expressed in CHO or HEK cells demonstrated the highest stabilities, with higher stability for the proBNP expressed in HEK cells. More than 90% of the initial immunoreactivity was observed for both proteins after 96 hours of incubation at 4° C.; 70% and 82% of initial immunoreactivity for proteins expressed in CHO and HEK cell lines, respectively, were observed after 96 hours of incubation at 25° C. At both temperatures, the stability of the antigen expressed in human HEK cells during long-term incubation was higher than the stability of the antigen expressed in CHO cells.

In conclusion, recombinant proBNPs demonstrated significantly increased stability of BNP immunoreactivity in comparison with synthetic BNP-32 molecules. Further, glycosylated recombinant proBNPs expressed in eukaryotic cell lines were significantly more stable than non-glycosylated proBNP expressed in E. coli.

The data disclosed herein, along with the additional disclosure, establishes that immunoassays of proBNP forms of Brain Natriuretic Peptide, also proBNP fragments and modified forms of proBNP, provide materials for preparations and methods for standardizing such assays in a manner that results in improved accuracy and reproducibility of the assay results. These improvements, in turn, lead to more accurate and reliable diagnoses of cardiovascular disease, such as congestive heart failure. In addition, the improved assays are useful for prognostic applications and the monitoring of treatment for patients exhibiting, or at risk of exhibiting, cardiovascular disease or symptoms characteristic thereof.

### Example 15

### Antibodies Directed to an Epitope Comprising Thr71 can Detect Only Small Portion of Endogenous proBNP

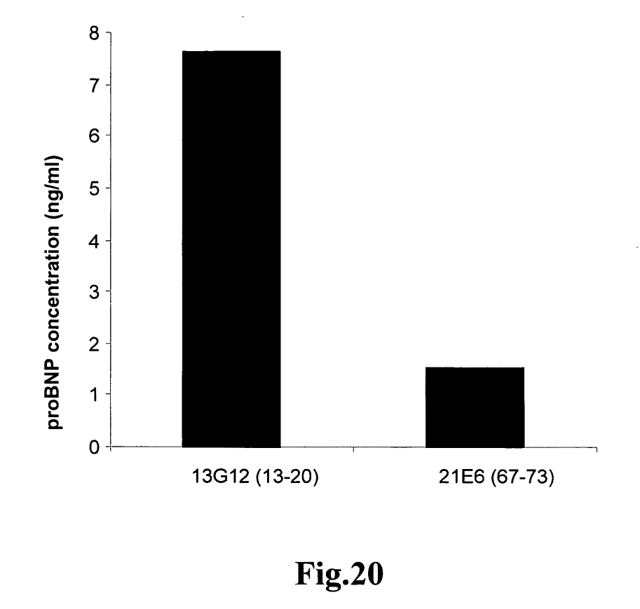

Two proBNP assays were used to quantify proBNP in a pooled sample of plasma from patients with heart failure (HF). In both assays the detection antibody was specific to the BNP portion of proBNP molecule (MAb 24C5 specific to peptide 11-22 of BNP molecule, or to peptide 87-98 of proBNP molecule), whereas the capture antibodies were different. Assay 1 utilized MAb 13G12 specific to the N-terminal portion of proBNP molecule (epitope 13-20) as a capture antibody. In Assay 2 MAb 21E6, specific to the epitope 67-73, comprising Thr71, is used as a capture antibody. Recombinant proBNP, expressed in E. coli (originally not glycosylated) was used in both assays as a calibrator. As it can be seen from FIG. 20, Assay 2 was able to detect only ⅕ of the antigen from that detected by Assay 1 in pooled HF plasma. This observation confirms our theory that Thr71 in endogenous proBNP is for the most part glycosylated, making major part of proBNP molecules “invisible” for Assay 2 utilizing the antibody directed to the epitope comprising Thr71.

Treatment of isolated endogenous proBNP by enzymes responsible for the deglycosylation of O-glycosylated proteins resulted in the significant (3-5-fold) growth of proBNP values detected by Assay 2, but almost no difference in antigen immunodetection before and after deglycosylation was observed for the Assay 2.

### Example 16

### Recombinant proBNP with Thr71Ala Mutation Expressed in Eukaryotic Cells is Almost Completely Processed to the NT-proBNP and BNP Fragments

Cells of line HEK 293 were transfected by two vectors, containing wild type of proBNP gene and proBNP gene with Thr71Ala mutation, respectively. The ratio of concentrations of two peptides—NT-proBNP and proBNP—in cell culture conditioned media was compared. It was demonstrated that in the case of wild type gene the major part of the proBNP (about 60%) remained unprocessed, whereas in the case of Thr71Ala mutation more than 80% of proBNP molecules were cleaved by endogenous protease forming NT-proBNP and BNP. Only small part of the antigen remained unprocessed.

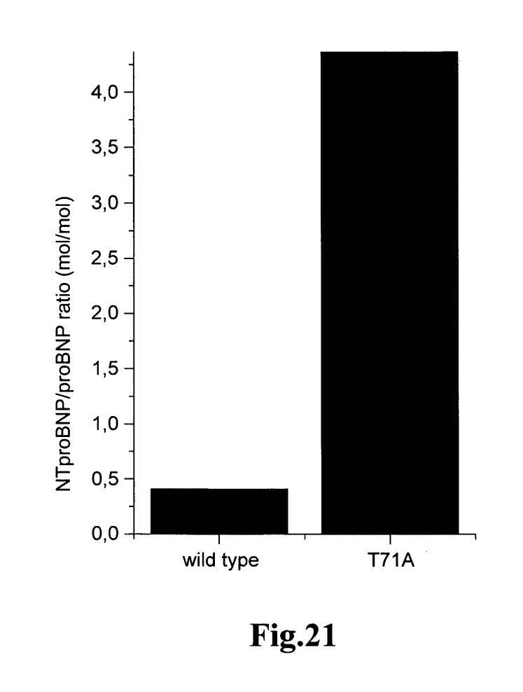

It was concluded that when Thr in position 71 is replaced by Ala, this site of proBNP molecule cannot be glycosylated. As a result, the site of proteolysis located between amino acid residues 76 and 77 in Thr71Ala mutant is not protected by glycosylation and is accessible for the proteases. FIG. 21 thus shows NT-proBNP/proBNP ratios in culture media of cells transfected by plasmids containing the wild type and Thr71Ala genes.

### Example 17

### Proteolysis of Glycosylated and Non-Glycosylated proBNP by Furin

It was shown that proBNP expressed by cells HEK 293 transfected by a plasmid containing the wild type of proBNP gene are heavily glycosylated and antibodies specific to the epitopes comprising Thr71 are almost unable to recognize such recombinant protein. Stability of heavily glycosylated proBNP expressed by HEK 293 and proBNP expressed by E. coli cells (not glycosylated) to protease furin was compared. Furin is considered to be exactly the enzyme which converts proBNP to BNP and NT-proBNP in human cardiac tissue.

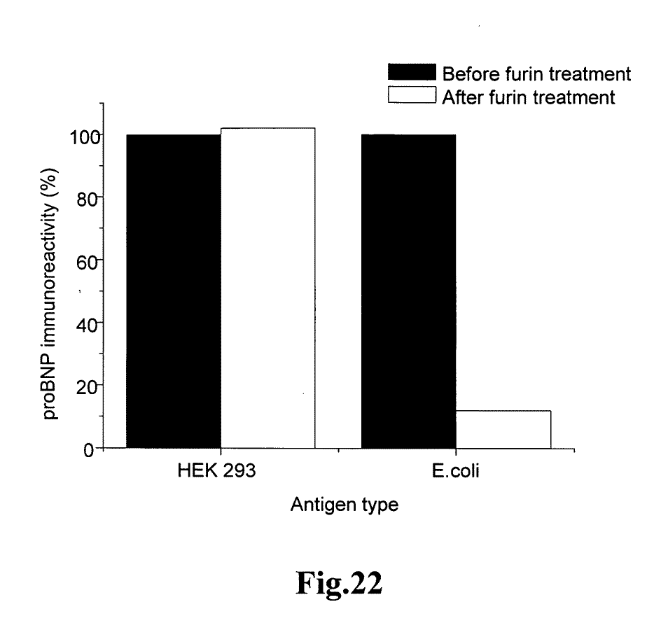

It was shown (FIG. 22) that furin is unable to cleave proBNP expressed in HEK 293 cells with Thr71 glycosylated, whereas proBNP expressed in E. coli cells, not glycosylated, was almost completely converted into BNP and NT-proBNP.

### Example 18

### Antibodies Specific to Region 63-76 Cannot Recognize proBNP Expressed in HEK 293 Cells, but can Recognize about 40% of proBNP Expressed in CHO-K1 Cells

Monoclonal antibodies (MAbs) specific to different regions of human proBNP molecules were from HyTest Ltd. MAbs epitopes corresponding to proBNP amino acid sequence are designated as subscript (24C5₈₇₋₉₈).

Human recombinant proBNP expressed in E. coli was used as calibrator in the immunoassay. The peptide was from HyTest Ltd.

Expression of Recombinant proBNP in HEK 293 and CHO-K1 Cell Lines

HEK 293 and CHO-K1 cells were transiently transfected with a plasmid containing gene encoding human proBNP using cationic lipid-based transfection reagent.

### Sandwich Immunofluorescent Assay (IFA)

Capture antibodies, 2 μg per well in 100 μL of PBS, were incubated in 96-well plates for 30 min at room temperature. After washing, 50 μL of tested sample or calibrator and 50 μL of detection antibodies labeled with stable europium (III) chelate in assay buffer were added. After 30 min incubation the plates were washed, then enhancement solution was added, and fluorescence was measured.

Study of Immunochemical Properties of proBNP Using Sandwich IFAs

ProBNP from HF conditioned media was characterized using eleven two-site MAbs combinations. Capture antibodies were specific to different epitopes covering whole NT-proBNP molecule (29D2₅₋₁₂, 21E3₁₃₋₂₀, 1D4₁₃₋₂₄, 5D3₂₈₋₄₅, 11D13₁₋₃₉, 16E6₃₄₋₃₉, 16D10₄₆₋₅₆, 15C4₆₃₋₇₁, 21E6₆₇₋₇₃, 24E11₆₇₋₇₆, 28F8₆₇₋₇₆). MAb 24C5₈₇₋₉₈ specific to the C-terminal part of proBNP was used as detection. Pooled conditioned media from transfected cells were used as a source of recombinant proBNP.

The results are shown in FIG. 23, demonstrating that antibodies specific to the region 63-76 cannot recognize proBNP expressed in HEK 293 cells, but can recognize about 40% of proBNP expressed in CHO-K1 cells.

### Example 19

### Immunochemical Activity Profiles of proBNP and NT-proBNP, and Effect of Deglycosylation on Immunoreactivity of proBNP

Monoclonal antibodies (MAbs) specific to different regions of human proBNP molecules were from HyTest. MAbs epitopes corresponding to proBNP amino acid sequence are designated as subscript (24C5₈₇₋₉₈).

Human recombinant NT-proBNP and pro-BNP expressed in E. coli were used as calibrators in immunoassays. Both peptides were from HyTest.

Extraction of Endogenous proBNP from HF Patients' Plasma

To prepare affinity matrix for proBNP extraction two MAbs specific to the C-terminal part of proBNP (MAbs 24C5₈₇₋₉₈ and 50E1₁₀₂-108) were coupled with Sepharose CL 4B.

Enzymatic Deglycosylation of proBNP

Endogenous proBNP extracted from plasma was incubated with either an enzyme mixture (endo-α-N-acetylgalactosaminidase, N-acetylneuraminate glycohydrolase, β-N-acetylhexosaminidase, β(1-4) galactosidase) or without enzymes for 1.5 hours at 37° C.

### Sandwich Immunofluorescent Assay (IFA)

Capture antibodies, 2 μg per well in 100 μL of PBS, were incubated in 96-well plates for 30 min at room temperature. After washing, 50 μL of tested sample or calibrator and 50 μL of detection antibodies labeled with stable europium (III) chelate in assay buffer were added. After 30 min incubation the plates were washed, then enhancement solution was added, and fluorescence was measured.

Studies of Immunochemical Properties of proBNP and NT-proBNP Using Sandwich IFAs

ProBNP from HF patients' plasma was characterized using eleven two-site MAbs combinations. Capture antibodies were specific to different epitopes covering whole NT-proBNP molecule (29D12₅₋₁₂, 21E3₁₃₋₂₀, 1D4₁₃₋₂₄, 5D3₂₈₋₄₅, 11D1₃₁₋₃₉, 16E6₃₄₋₃₉, 16D10₄₆₋₅₆, 15C4₆₃₋₇₁, 21E6₆₇₋₇₃, 24E11₆₇₋₇₆ and 28F8₆₇₋₇₆). MAb 24C5₈₇₋₉₈ specific to the C-terminal part of proBNP was used as detection. Pooled plasma from 12 HF patients was used as a source of endogenous (recombinant) proBNP. ProBNP treated and non-treated with glycosidases mixture was analyzed by IFAs described above. For NT-proBNP measurements pooled plasma was passed through affinity matrix to remove proBNP. The same set of capture antibodies was used for NT-proBNP measurements. Detection antibody 24E11₆₇₋₇₆ was used to form pairs with capture MAbs specific to region 5-24 of NT-proBNP, whereas detection MAb 13G12₁₃₋₂₀ was used in combinations with capture antibodies specific to region 28-76 of NT-proBNP.

The results are given in FIGS. 24A and 24B. When comparing the immunoreactivity profiles of proBNP and NT-proBNP from HF patients' plasma using MAbs specific to different regions of NT-proBNP molecule, it was found that the profile of immunochemical activity for both molecules was similar with the exception for the region 61-76 located close to the proBNP cleavage site 76R↓S77 (FIG. 24A). The region 61-76 of endogenous proBNP was inaccessible to specific MAbs, whereas the same antibodies recognized endogenous NT-proBNP with high efficiency. When proBNP extracted from HF patients' plasma was treated with mixture of O-specific glycosidases, it was found that deglycosylation resulted in 2-2.3-fold increase of immunoreactivity of proBNP in the assays using MAbs specific to the region 61-76 (FIG. 24B). So it was concluded that region located close to the cleavage site is glycosylated in endogenous proBNP in comparison with NT-proBNP.

### Example 20

### C-Terminal Part of Recombinant NT-proBNP is not Glycosylated but the Same Region of proBNP Molecule is Heavily Glycosylated

Human recombinant NT-proBNP and proBNP expressed in E. coli (originally nonglycosylated polypeptides) were from HyTest. The antigens were used as calibrators in immunoassays.

Monoclonal antibodies (MAbs) specific to different regions of human proBNP were from HyTest. MAbs epitopes corresponding to proBNP amino acid sequence are designated as subscript (24C5₈₇₋₉₈).

Sandwich immunofluorescent assay (IFA). Capture antibodies, 2 μg per well in 100 μL of PBS, were incubated in immunoassay plates for 30 min at room temperature. After washing, 50 μL of tested sample or calibrator and 50 μL of detection antibodies labeled with stable europium (III) chelate in assay buffer were added. After incubation for 30 min at room temperature, the plates were washed, then enhancement solution was added, and fluorescence was measured.

Studies of immunochemical properties of proBNP and NT-proBNP using sandwich IFAs. ProBNP and NT-proBNP from conditioned media (HEK 293 cells transiently transfected with human proBNP cDNA) were characterised using eleven two-site MAbs combinations. Capture antibodies were specific to different epitopes covering whole NT-proBNP molecule (29D1₂₅₋₁₂, 21E3₁₃₋₂₀, 1D4₁₃₋₂₄, 5D3₂₈₋₄₅, 11D1₃₁₋₃₉, 16F6₃₄₋₃₉, 16D10₄₆₋₅₆, 15C4₆₃₋₇₁, 21E6₆₇₋₇₃, 24E11₆₇₋₇₆ and 28F8₆₇₋₇₆). In proBNP assays MAb 24C5₈₇₋₉₈ specific to the C-terminal part of proBNP was used as detection. For NT-proBNP measurements conditioned media was passed through affinity matrix to remove proBNP. The same panel of capture antibodies was used for NT-proBNP measurements. Detection antibody 24E11₆₇₋₇₆ was used to form pairs with capture MAbs specific to region 5-24 of NT-proBNP, whereas detection MAb 13G12₁₃₋₂₀ was used in combinations with capture antibodies specific to region 28-76 of NT-proBNP.

Cell culturing and transfection. HEK 293 (human embryonic kidney cell line) was obtained from the American Type Culture Collection. Cells were cultured in DMEM supplemented with 10% FBS. Transient transfection of cells was performed using cationic lipid-based transfection reagent.

The results are given in FIG. 25. When proBNP was expressed in HEK 293 cells by means of transient transfection, both proBNP and products of its processing—BNP and NT-proBNP—were detected in conditioned media. We compared the immunoreactivity profiles of recombinant proBNP and NT-proBNP using MAbs specific to different regions of proBNP molecule. Similarly to endogenous peptides, in recombinant NT-proBNP C-terminal part of the molecule was not glycosylated (the region was accessible for region-specific MAbs), whereas the same region of proBNP molecule was heavily glycosylated (the region was inaccessible to region-specific MAbs) (FIG. 25).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the features set forth herein.

### REFERENCES

Cowie M R, Mendez G F. BNP and congestive heart failure. Prog. Cardiovasc. Dis. 2002; 44:293-321.
Crimmins D L. Human N-terminal proBNP is a monomer. Clin. Chem. 2005; 51:1035-1038.
Giuliani I, Rieunier F, Larue C, et al. Assay for measurement of intact B-type natriuretic peptide prohormone in blood. Clin. Chem. 2006; 52:1054-1061.
Hughes D, Talwar S, Squire I B, Davies J E, Ng L L. An immunoluminometric assay for N-terminal pro-brain natriuretic peptide: development of a test for left ventricular dysfunction. Clin. Sci. (Lond) 1999; 96:373-380.
Hunt P J, Espiner E A, Nicholls M G, Richards A M, Yandle T G. The role of the circulation in processing pro-brain natriuretic peptide (proBNP) to amino-terminal BNP and BNP-32. Peptides 1997; 18:1475-1481.
Karl J, Borgya A, Gallusser A, et al. Development of a novel, N-terminal-proBNP (NT-proBNP) assay with a low detection limit. Scand. J. Clin. Lab. Invest. Suppl. 1999; 230:177-181.
Luchner et al., Hypertension, 2002 January; 39 (1): 99-104
Mair J, Hammerer-Lercher A, Puschendorf B. The impact of cardiac natriuretic peptide determination on the diagnosis and management of heart failure. Clin. Chem. Lab. Med. 2001; 39:571-88.
Pandey K N. Biology of natriuretic peptides and their receptors. Peptides 2005; 26:901-932.
Sawada Y, Suda M, Yokoyama H, Kanda T, Sakamaki T, Tanaka S, et al. Stretch-induced hypertrophic growth of cardiocytes and processing of brain-type natriuretic peptide are controlled by proprotein-processing endoprotease furin. J. Biol. Chem. 1997; 272:20545-20554.
Schagger H, von Jagow G. Tricine-sodium dodecyl sulfate-polyacrylamide gel electrophoresis for the separation of proteins in the range from 1 to 100 kDa. Anal. Biochem. 1987; 166:368-379.
Schellenberger U, O'Rear J, Guzzetta A, Jue R A, Protter A A, Pollitt N S. The precursor to B-type natriuretic peptide is an O-linked glycoprotein. Arch. Biochem. Biophys. 2006; 451:160-166.
Seferian K R, Tamm N N, Semenov A G, Tolstaya A A, Koshkina E V, Krasnoselsky M I, et al. Immunodetection of glycosylated NT-proBNP circulating in human blood. Clin Chem 2008; 54:866-873.
Seidler T, Pemberton C, Yandle T, Espiner E, Nicholls G, Richards M. The amino terminal regions of proBNP and proANP oligomerise through leucine zipper-like coiled-coil motifs. Biochem. Biophys. Res. Commun. 1999; 255:495-501.
Troughton et al., Lancet, 2000 Apr. 1, 355 (9210):1126-1130.
Wu et al., Clin Chem. 2004 May; 50 (5): 867-873.
Yan W, Wu F, Morser J, Wu Q. Corin, a transmembrane cardiac serine protease, acts as a pro-atrial natriuretic peptide-converting enzyme. Proc Natl Acad Sci USA 2000; 97:8525-8529.

1-40. (canceled)

41. An antibody, which specifically recognizes endogenous glycosylated NT-proBNP or proBNP or a fragment thereof, and which does not recognize deglycosylated NT-proBNP or proBNP or a fragment thereof, or a fragment of such an antibody.

42. An antibody, which recognizes endogenous glycosylated NT-proBNP or proBNP or a fragment thereof with higher affinity than said antibody recognizes a corresponding deglycosylated protein or a fragment thereof, or a fragment of such an antibody.

43. A pro-Brain Natriuretic Peptide (proBNP) standard or calibration preparation for use in a method for detecting BNP having the sequence given in SEQ ID NO:4, or its fragments or molecules comprising this sequence or part of it in a sample, wherein the standard preparation comprises an isolated or recombinant or synthetic proBNP having the amino acid sequence given in SEQ ID NO:1, or a fragment or modification thereof, in glycosylated form, in combination with at least one diluent.

44. The proBNP standard or calibration preparation according to claim 43, wherein the proBNP or the fragment or modification thereof is expressed in eukaryotic cells.

45. The proBNP standard or calibration preparation according to claim 43, wherein Thr71 of the proBNP sequence as set forth in SEQ ID NO:1 is glycosylated.

46. The proBNP standard or calibration preparation according to claim 43, wherein the proBNP or the fragment or modification thereof is a synthetic polypeptide.

47. An NT-proBNP or proBNP standard or calibration preparation comprising a glycosylated NT-proBNP or proBNP or a fragment thereof.

48. A recombinant or synthetic proBNP molecule, wherein at least one of the amino acid residues 66 to 76 of SEQ ID NO:1 is modified, or a fragment of said proBNP molecule.

49. The proBNP molecule or a fragment thereof according to claim 48, which is modified by glycosylation or pegylation.

50. A diagnostic method for assaying NT-proBNP or proBNP or a fragment thereof in a patient sample, comprising quantitative, semiquantitative or qualitative determination of the NT-proBNP or proBNP content of the sample using an antibody according to claim 41.

51. The diagnostic method according to claim 50, further comprising preparing a calibration curve using as the standard a preparation of an endogenous glycosylated NT-proBNP or proBNP, isolated from vertebrate tissue or body fluid.

52. An immunoassay method for detection or quantification of NT-proBNP in a sample employing an antibody combination, wherein one antibody is specific to a fragment of NT-proBNP which comprises Thr71, wherein the amino acid sequence of said fragment is given as residues 65 to 76 of SEQ ID NO:3, and another antibody is specific to the N-terminal portion of NT-proBNP which comprises the amino acid residues 1 to 30 of SEQ ID NO:3.

53. An immunoassay method for measuring the ratio of NT-proBNP/proBNP or proBNP/NT-proBNP concentrations in a sample, comprising
(a) measuring NT-proBNP concentration in an immunoassay method employing an antibody combination, wherein one antibody is specific to a fragment of NT-proBNP which comprises Thr71, wherein the amino acid sequence of said fragment is given as residues 65 to 76 of SEQ ID NO:3, and another antibody is specific to the N-terminal portion of NT-proBNP which comprises the amino acid residues 1 to 30 of SEQ ID NO:3,
(b) measuring the proBNP concentration in an immunoassay method, and
(c) comparing the concentration values obtained from steps (a) and (b) in whichever order to obtain the ratio of NT-proBNP/proBNP or proBNP/NT-proBNP concentrations.

54. The immunoassay method according to claim 53, wherein step (b) for measuring the proBNP concentration in an immunoassay method is carried out employing an antibody combination, wherein one antibody is specific to the N-terminal portion of proBNP comprising the amino acid residues 1 to 30 of SEQ ID NO:1, and another antibody is specific to the BNP portion of proBNP comprising the amino acid residues 77 to 108 of SEQ ID NO:1.

55. A diagnostic method for assaying NT-proBNP or proBNP in a sample of a patient, comprising
(a) deglycosylating endogenous NT-proBNP or proBNP contained in the sample, and
(b) determining the NT-proBNP or proBNP content of the sample using an antibody or an aptamer specific to NT-proBNP or proBNP.

56. A diagnostic immunoassay method for determining immunoreactivity of BNP having the sequence given in SEQ ID NO:4, or its fragments, or molecules comprising this sequence or part of it in a body fluid sample of a patient, comprising:
(a) measuring BNP immunoreactivity in a sample;
(b) preparing a standard curve using as a standard a polypeptide selected from the group consisting of an isolated or recombinant or synthetic proBNP having the amino acid sequence given in SEQ ID NO:1, an isolated or recombinant or synthetic proBNP fragment and an isolated or recombinant or synthetic modified proBNP; and
(c) comparing the value of the BNP immunoreactivity obtained in step (a) to the standard curve produced in step (b) in order to quantify the BNP immunoreactivity in the sample.

57. An immunoassay kit for diagnostic assay of NT-proBNP or proBNP or a fragment thereof in a sample of a patient, the kit comprising
(a) a monoclonal or polyclonal antibody having the same specificity as an antibody of claim 41,
(b) a detectable label, and
(c) a standard or calibrator preparation according to claim 47.

58. An immunoassay kit for diagnostic measurement of BNP immunoreactivity in a sample of a patient, the kit comprising:
a monoclonal or polyclonal antibody specific to BNP;
a detectable label; and
as a standard for preparing a standard curve, a polypeptide selected from the group consisting of an isolated or recombinant or synthetic proBNP comprising the amino acid sequence given in SEQ ID NO:1 or an isolated or recombinant or synthetic proBNP fragment in glycosylated form.

59. A method of treatment of a disorder, comprising administering to a patient in need of such treatment an efficacious amount of a mutated proBNP polypeptide, from which Thr71 has been deleted or in which Thr71 residue has been changed to any other amino acid residue, or a nucleic acid encoding a proBNP mutant, from which a fragment encoding Thr71 has been deleted or in which a fragment encoding Thr71 residue has been changed to a fragment encoding any other amino acid residue.

60. The method according to claim 59, wherein the disorder is heart failure.

61. A recombinant proBNP polypeptide, wherein the Thr71 residue is glycosylated.

62. The recombinant proBNP polypeptide according to claim 61, wherein the polypeptide is expressed in eukaryotic cells.

63. A recombinant or synthetic proBNP polypeptide having at least one modification in any of its amino acid residues 66 to 76.

64. The recombinant or synthetic proBNP polypeptide according to claim 63, wherein the modification is Thr71 Ala (SEQ ID NO:5).


SEQUENCE LISTING (5 sequences)

SEQ ID NO: 1 - protein, 108 aa, Homo sapiens
  MISC_FEATURE: proBNP
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Homo sapiens
  MISC_FEATURE: preproBNP
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 76 aa, Homo sapiens
  MISC_FEATURE: NT-proBNP
HPLGSPGSASDLETSGLQEQRNHLQGKLSELQVEQTSLEPLQESPRPTGVWKSREVATEGIRGHRKMVLYTLRAPR

SEQ ID NO: 4 - protein, 32 aa, Homo sapiens
  MISC_FEATURE: BNP
SPKMVQGSGCFGRKMDRISSSSGLGCKVLRRH

SEQ ID NO: 5 - protein, 108 aa, Artificial sequence
  Thr71Ala mutation of proBNP
HPLGSPGSASDLETSGLQEQRNHLQGKLSELQVEQTSLEPLQESPRPTGVWKSREVATEGIRGHRKMVLYALRAPRSPKMVQGSGCFGRKMDRISSSSGLGCKVLRRH